(19) United States
(12) Patent Application Publication
(10) Pub. No.: US 2023/0203490 A1
(43) Pub. Date: Jun. 29, 2023

(54) UTILIZING RNA INTERFERENCE AGAINST SARS-COV-2

(71) Applicant: ELEVEN THERAPEUTICS LTD., Tel Aviv (IL)

(72) Inventor: Yaniv ERLICH, Raanana (IL)

(21) Appl. No.: 17/923,586

(22) PCT Filed: May 5, 2021
(86) PCT No.: PCT/IL2021/050511

§ 371 Date: Nov. 6, 2022

Related U.S. Application Data
(60) Provisional application No. 63/138,843, filed on Jan. 19, 2021.
(60) Provisional application No. 63/021,399, filed on May 7, 2020.

(51) Int. Cl. C12N 15/113; A61P 31/14; A61P 11/00

(52) U.S. Cl. CPC C12N 15/1131; A61P 31/14; A61P 11/00; C12N 2310/14

(57) ABSTRACT

An RNAi molecule, the RNAi molecule, vector and composition including the RNAi molecule and method of using same, wherein the RNAi molecule includes a dsRNA sequence of from about 15 to about 60 base pairs, capable of decreasing expression of a SARS-CoV-2 gene.

### BACKGROUND

Covid-19 has been one of the world's worst pandemic in modern times. As of early April 2020, over 1.6 million people have been affected and more than 100,000 individuals have succumbed to the disease. Beyond the human toll, the pandemic also created a massive economic turmoil due to unprecedented steps took by Governments to control the disease. Recent epidemiological modeling² suggests that the SARS-CoV-2 pandemic will undergo multiple waves of infections worldwide over the next years. These waves are likely to continue until a level of herd immunity greater than 60% is achieved (through both natural infection and vaccination). Even then, the virus will not be eliminated and will exhibit a typical seasonal pattern with local effects.

siRNA are short, double stranded RNA molecules between 18 to 25nt. When entering into cells, these molecules are loaded into the RNA interference (RNAi) machinery, which endogenously exist in human cells. The RNAi machinery scavenges the cytoplasm for long RNA molecules that specifically exhibit sequence complementarity to the loaded siRNA sequences. When it identifies a target RNA molecule with such complementarity, it silences its expression and prevents protein translation. shRNAs are quite similar but instead of administrating double stranded RNA, these molecules are typically administered as part of a genetic engineering step where they are introduced into the host genome, for example by lentivirus infection. The shRNA behaves as a new gene in the genome that is transcribed by the host RNA machinery into a single piece of RNA molecule. The shRNA is folded into a hairpin structure and recognized by a series of endogenous enzymes that cut the structure until a short double stranded RNA is created. One strand of the double stranded RNA is loaded into the RNAi machinery and can repress long reverse complement RNA molecules.

### SUMMARY OF THE INVENTION

There is provided herein siRNAs and/or shRNA (commonly referred to herein as RNAi molecules), compositions comprising the RNAi molecules and methods for utilizing same for preventing infection, reducing spread and/or reducing viral load in a subject in need thereof.

Advantageously, the hereindisclosed RNAi molecules are highly potent and thus retain high efficacy even at small doses, thereby reducing the cost of the final therapeutic product as well as minimizing risk off cross-reactivity against transcripts derived from the host genome. According to some embodiments, the composition may include a “cocktail” of RNAi molecules. This may enable high optionally improved efficacy. According to some embodiments, each of the RNAi molecules of the cocktail may be provided at a lower dose than that required if provided as a single molecule, thereby further increasing the safety of the therapeutic product.

As a further advantage, the hereindisclosed RNAi molecules are conserved among a wide range of SARS-CoV2 strains thus providing broad-range efficacy.

According to some embodiments, the herein provided compositions may have an essentially on the spot efficacy (e.g. within an hour or even half an hour after administration. This may advantageously enable the use of the composition for first line protection. As a non-limiting example, a paramedic receiving a call to a patient showing Covid-19 symptoms may take the composition prior to arrival so as to minimize risk of being infected by the patient. As another non-limiting example, medical workers or other care-givers may be administered with the composition prior to enter covid-19 infected facilities. Advantageously, the hereindisclosed RNAi molecules are stable e.g. for approximately 12 hours, thus providing protection during approximately the same amount of time. Subsequently the RNAi molecules are self-degraded thus having no or few long-term implications for the subject administered therewith.

According to some embodiments, there is provided an isolated ribonucleic acid interfering (RNAi) molecule, comprising a dsRNA sequence of from about 15 to about 60 base pairs, capable of decreasing expression of a SARS-CoV-2 gene, wherein a guide strand of the dsRNA comprises at least one nucleotide sequence having at least 80% sequence homology to any one of SEQ ID NO: 1-14425.

According to some embodiments, there is provided an isolated ribonucleic acid interfering (RNAi) molecule, comprising a dsRNA sequence of from about 15 to about 60 base pairs, capable of decreasing expression of a SARS-CoV-2 gene, wherein a guide strand of the dsRNA comprises at least one nucleotide sequence having at least 80% sequence homology to any one of SEQ ID NO: 1-99.

According to some embodiments, there is provided an isolated ribonucleic acid interfering (RNAi) molecule, comprising a dsRNA sequence of from about 15 to about 60 base pairs, capable of decreasing expression of the NSP8 gene of SARS-CoV-2, wherein a guide strand of the dsRNA comprises at a nucleotide sequence having at least 80% sequence homology to any one of SEQ ID NO: 3-10.

According to some embodiments, there is provided an isolated ribonucleic acid interfering (RNAi) molecule, comprising a dsRNA sequence of from about 15 to about 60 base pairs, capable of decreasing expression of the NSP8 gene of SARS-CoV-2, wherein a guide strand of the dsRNA comprises at a nucleotide sequence having at least 80% sequence homology to any one of SEQ ID NO: 4 and 6-9.

According to some embodiments, there is provided an isolated ribonucleic acid interfering (RNAi) molecule, comprising a dsRNA sequence of from about 15 to about 60 base pairs, capable of decreasing expression of the helicase gene of SARS-CoV-2, wherein a guide strand of the dsRNA comprises at a nucleotide sequence having at least 80% sequence homology to any one of SEQ ID NO: 11-29 and 82.

According to some embodiments, there is provided an isolated ribonucleic acid interfering (RNAi) molecule, comprising a dsRNA sequence of from about 15 to about 60 base pairs, capable of decreasing expression of the helicase gene of SARS-CoV-2, wherein a guide strand of the dsRNA comprises at a nucleotide sequence having at least 80% sequence homology to any one of SEQ ID NO: 12-13 and 99. According to some embodiments, there is provided an isolated ribonucleic acid interfering (RNAi) molecule, comprising a dsRNA sequence of from about 15 to about 60 base pairs, capable of decreasing expression of the helicase gene of SARS-CoV-2, wherein a guide strand of the dsRNA comprises at a nucleotide sequence having at least 80% sequence homology to SEQ ID NO: 13.

According to some embodiments, there is provided an isolated ribonucleic acid interfering (RNAi) molecule, comprising a dsRNA sequence of from about 15 to about 60 base pairs, capable of decreasing expression of the RNA dependent RNA polymerase gene of SARS-CoV-2, wherein a guide strand of the dsRNA comprises at a nucleotide sequence having at least 80% sequence homology to any one of SEQ ID NO: 30-46, 80 and 81.

According to some embodiments, there is provided an isolated ribonucleic acid interfering (RNAi) molecule, comprising a dsRNA sequence of from about 15 to about 60 base pairs, capable of decreasing expression of the RNA dependent RNA polymerase gene of SARS-CoV-2, wherein a guide strand of the dsRNA comprises at a nucleotide sequence having at least 80% sequence homology to any one of SEQ ID NO: 33-35 and 46.

According to some embodiments, there is provided an isolated ribonucleic acid interfering (RNAi) molecule, comprising a dsRNA sequence of from about 15 to about 60 base pairs, capable of decreasing expression of the Spike gene of SARS-CoV-2, wherein a guide strand of the dsRNA comprises at a nucleotide sequence having at least 80% sequence homology to any one of SEQ ID NO: 47-71 and 83-88.

According to some embodiments, there is provided an isolated ribonucleic acid interfering (RNAi) molecule, comprising a dsRNA sequence of from about 15 to about 60 base pairs, capable of decreasing expression of the Spike gene of SARS-CoV-2, wherein a guide strand of the dsRNA comprises at a nucleotide sequence having at least 80% sequence homology to any one of SEQ ID NO: 53, 54, 60, 62, 66, 68, 69, 83 and 84.

According to some embodiments, there is provided an isolated ribonucleic acid interfering (RNAi) molecule, comprising a dsRNA sequence of from about 15 to about 60 base pairs, capable of targeting the reverse complement of the leader of SARS-CoV-2, wherein a guide strand of the dsRNA comprises at a nucleotide sequence having at least 80% sequence homology to any one of SEQ ID NO: 72-78.

According to some embodiments, there is provided an isolated ribonucleic acid interfering (RNAi) molecule, comprising a dsRNA sequence of from about 15 to about 60 base pairs, capable of targeting the reverse complement of the leader of SARS-CoV-2, wherein a guide strand of the dsRNA comprises at a nucleotide sequence having at least 80% sequence homology to any one of SEQ ID NO: 76-78.

According to some embodiments, the first strand and the second strand of the dsRNA include at least 14-25 contiguous complementary base pairs.

According to some embodiments, the first and/or the second strands comprise a 3′ single-stranded overhang. According to some embodiments, the overhang comprises at least two nucleotides. According to some embodiments, the dsRNA comprises a nucleotide analogue.

According to some embodiments, the at least one nucleotide sequence has 100% sequence homology to any one of SEQ ID NO: 1-14425.

According to some embodiments, the at least one nucleotide sequence has 100% sequence homology to any one of SEQ ID NO 1-99.

According to some embodiments, the RNAi molecule is a siRNA. According to some embodiments, the RNAi molecule is a shRNA.

According to some embodiments, there is provided an RNAi-inducing vector comprising a nucleotide sequence encoding at least one RNAi molecule and a promoter transcriptionally associated with the nucleotide sequence, the RNAi molecule comprising a dsRNA sequence of from about 15 to about 60 base pairs, capable of decreasing expression of a SARS-CoV-2 gene, wherein the RNAi molecule comprises at least one nucleotide sequence having at least 80% sequence homology to any one of SEQ ID NOs: 1-80 and 89-99, or SEQ ID Nos: 3-10; or 4 and 6-9; or SEQ ID Nos: 11-29 and 82; or SEQ ID Nos: 12-13; or SEQ ID Nos: 30-46, 80 and 81; or SEQ ID Nos: 33-35 and 46; or SEQ ID Nos: 47-71 and 83-88; or SEQ ID Nos: 53, 54, 60, 62, 66, 68, 69, 83 and 84; or SEQ ID Nos: 72-78; or SEQ ID Nos: 76-78; or SEQ ID Nos 89-99, SEQ ID Nos 9353, 9452, 9549, and 14426-14428. Each possibility is a separate embodiment.

According to some embodiments, the first strand and the second strand of the dsRNA comprises at least 14-25 contiguous complementary base pairs According to some embodiments, wherein the dsRNA comprises a nucleotide analogue.

According to some embodiments, the promoter is a constitutively active promoter, an inducible promoter and/or tissue specific promoter.

According to some embodiments, the transcription vector is any one of an adeno-viral vector or a lentiviral vector.

According to some embodiments, there is provided a composition comprising the one or more of the hereindisclosed RNAi molecules or a hereindisclosed vector including the RNAi molecule, and a suitable transport vehicle and/or carrier.

According to some embodiments, the composition comprises at least two different RNAi molecules, wherein the first strand of each of the at least two RNAi molecules has 80% sequence homology to different ones of any one of SEQ ID NOs: 1-80 and 89-99, or SEQ ID Nos: 3-10; or 4 and 6-9; or SEQ ID Nos: 11-29 and 82; or SEQ ID Nos: 12-13; or SEQ ID Nos: 30-46, 80 and 81; or SEQ ID Nos: 33-35 and 46; or SEQ ID Nos: 47-71 and 83-88; or SEQ ID Nos: 53, 54, 60, 62, 66, 68, 69, 83 and 84; or SEQ ID Nos: 72-78; or SEQ ID Nos: 76-78; or SEQ ID Nos 89-99. Each possibility is a separate embodiment.

According to some embodiments, the carrier is aqueous solution.

According to some embodiments, the composition is suitable for administration through aerosol.

According to some embodiments, the transport vehicle is a liposome or a lipid nanoparticle.

According to some embodiments, the composition is formulated for oral and/or nasal administration. According to some embodiments, the composition is formulated for administration via inhalation. According to some embodiments, the composition is formulated for intranasal and/or intrabuccal administration.

According to some embodiments, there is provided a method for preventing infection, reducing viral load, attenuating and/or inhibiting spread of SARS-CoV-2 infection, the method comprising administering to a subject the hereindisclosed RNAi molecule or the hereindisclosed composition comprising the RNAi molecule.

According to some embodiments, the subject is a subject infected with the SARS-CoV-2. According to some embodiments, the subject is a care-giver of a patient infected with the SARS-CoV-2 or suspected as being infected with SARS-CoV-2. According to some embodiments, the subject is an individual having high risk of severe illness if infected with SARS-CoV-2. According to some embodiments, the subject is an intermediate host.

According to some embodiments, the method comprises administering 150 mg siRNA/day.

According to some embodiments, there is provided any of the herein disclosed RNAi molecules or composition comprising same for use in preventing infection, reducing viral load, attenuating and/or inhibiting spread of SARS-CoV-2 infection.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures so that it may be more fully understood.

FIG. 1 is a flowchart of a method for identifying potential RNAi molecules; according to some embodiments.

FIG. 2 is a flowchart of a method for identifying potential RNAi molecules; according to some embodiments.

FIG. 3 is a flowchart of a method for identifying potential RNAi molecules; according to some embodiments.

FIG. 4 shows representative FACS results and gating strategy.

FIG. 5A-FIG. 5C show representative FACS results of cells transfected with a control siRNA (negative control), target siRNA and siRNA against GFP (positive control), respectively.

FIG. 6A and FIG. 6B show the mCherry+GFP/GFP+ ratio (indicative of siRNAs efficiency), obtained for siRNAs targeting the helicase of SARS-COV2, utilizing 1 nM, 500 pM and 80 pM siRNA.

FIG. 7A and FIG. 7B show the mCherry+GFP/GFP+ ratio (indicative of siRNAs efficiency), obtained for various siRNAs targeting the transcript of NSP8 of SARS-COV2, utilizing 1nM, 500 pM and 80 pM siRNA.

FIG. 8A and FIG. 8B show the mCherry+GFP/GFP+ ratio (indicative of siRNAs efficiency), obtained for various siRNAs targeting the transcript of RNA dependent RNA polymerase of SARS-COV2, utilizing 1 nM, 500 pM and 80 pM siRNA.

FIG. 9A and FIG. 9B and 10 show the mCherry+GFP/GFP+ ratio (indicative of siRNAs efficiency), obtained for various siRNAs targeting the transcript of the S-protein of SARS-COV2, utilizing 1 nM, 500 pM and 80 pM siRNA.

FIG. 11A and FIG. 11B show the mCherry+GFP/GFP+ ratio (indicative of siRNAs efficiency), obtained for various siRNAs targeting the Reverse Complement of the transcript of leader protein of SARS-COV2, utilizing 1 nM, 500 pM and 80 pM siRNA.

FIG. 12A and FIG. 12B, show the IC50 of fluorescence of a siRNAs targeting NSP8 of SARS-COV2 at different concentrations of the siRNAs.

FIG. 13A and FIG. 13B, show the IC50 of fluorescence of various siRNAs targeting the various regions of SARS-COV2 genome, at different concentrations of siRNAs.

FIG. 14, shows live virus attenuation results obtained for siRNAs targeting the transcript of the spike of Sars-COV2 after challenging Vero E6 cells with 600 TCID 50 of SARS-CoV-2. The level of viral load was obtained via qPCR by a Allplex™ SARS-CoV-2 Assay manufactured by Seegene. The Orflab expression levels are denoted as “genomic region” and the average expression of the “E” and the “N” gene are denoted as “subgenomic region”. The level of 100% corresponds to the viral load detected in a control condition transfected with siRNA against eGFP.

FIG. 15 shows live virus attenuation results obtained for siRNAs targeting the transcript of NSP8 (SEQ ID NO: 7), RNA dependent RNA polymerase (SEQ ID NO: 48) alone and in a combination.

FIG. 16A and FIG. 16B, viral load after treatment of cells, infected with SARS-COV2, with various siRNAs recovered in the screen.

FIG. 17A and FIG. 17B, viral load after treatment of cells, infected with 6000 TCID50 of SARS-COV2, with various siRNA cocktails.

FIG. 17C and FIG. 17D, viral load after treatment of cells, infected with 600 TCID50 of SARS-COV2, with various siRNA cocktails.

FIG. 17E and FIG. 17F, viral load after treatment of cells, infected with 60 TCID50 of SARS-COV2, with various siRNA cocktails.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

For convenience, certain terms used in the specification, examples, and appended claims are collected here. Unless otherwise defined, all technical and scientific terms used herein have the same meaning as commonly understood by one of ordinary skill in the art to which this invention pertains.

As used herein, a “nucleotide” comprises a nitrogenous base, a sugar molecule, and a phosphate group. A nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, locked nucleic acids, arabinose, and hexose). According to some embodiments, the sugar and/or phosphate groups may be modified to include a peptide bond, so as to obtain a Peptide Nucleotide Acid (PNA).

As used herein, the term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g. by DNA replication or transcription of DNA or RNA, respectively). DNA and RNA can also be chemically synthesized. RNA can be post-transcriptionally modified. The terms “target mRNA” and “target transcript” are synonymous as used herein.

As used herein, the term “RNA interference” (“RNAi”) refers to selective intracellular degradation of RNA (also referred to as gene silencing). As used herein a RNAi molecule may collectively refer to small interfering RNAs and short hairpin RNA.

As used herein, the term “small interfering RNA” (“siRNA”), also referred to in the art as “short interfering RNAs,” refers to an RNA (or RNA analog) comprising between about 10-60 or 15-25 nucleotides (or nucleotide analogs) that is capable of directing or mediating RNA interference. Generally, as used herein the term “siRNA” refers to double stranded siRNA (as compared to single stranded or antisense RNA). In certain embodiments, the 3′ end of the RNAi molecules may include additional nucleotides that create an overhang, such as “TT”.

As used herein, the term “short hairpin RNA” (“shRNA”) refers to an siRNA (or siRNA analog) precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAi (as described for siRNA duplexes), and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are thought to be processed into siRNAs by the conserved cellular RNAi machinery. shRNAs are capable of inhibiting expression of a target transcript that is complementary to a portion of the shRNA (referred to as the antisense or guide strand of the shRNA). In general, the features of the duplex formed between the guide strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group.

As used herein, the term “RNAi-inducing vector” includes a vector whose presence within a cell results in transcription of one or more RNAs that self-hybridize or hybridize to each other to form an RNAi molecule. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an RNAi molecule. In general, the vector comprises a nucleic acid operably linked to expression signal(s) so that one or more RNA molecules that hybridize or self-hybridize to form an RNAi molecule is transcribed when the vector is present within a cell. Use of the term “induce” is not intended to indicate that the RNAi agent necessarily activates or upregulates RNAi in general but simply indicates that presence of the vector within a cell results in production of an RNAi agent within the cell, leading to an RNAi-mediated reduction in expression of an RNA to which the agent is targeted.

An RNAi-inducing entity is considered to be targeted to a target transcript for the purposes described herein if (1) the agent comprises a strand that is substantially complementary to the target transcript over 15-29 nucleotides, e.g., 15, more preferably at least about 17, yet more preferably at least about 18 or 19 to about 21-23 or 24-29 nucleotides. For example, in various embodiments of the invention the agent comprises a strand that has at least about 70%, preferably at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, e.g., over a window of evaluation of at least 15, more preferably at least about 17, yet more preferably at least about 18 or 19 to about 21-23 or 24-29 nucleotides in length; or (2) one strand of the RNAi agent hybridizes to the target transcript under stringent conditions for hybridization of small (<50 nucleotide) RNA molecules in vitro and/or under conditions typically found within the cytoplasm or nucleus of mammalian cells.

As used herein, the term “complementary” refer to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation). A degree of complementarity of two nucleic acids or portions thereof may be evaluated by determining the total number of nucleotides in both strands that form complementary base pairs as a percentage of the total number of nucleotides over a window of evaluation when the two nucleic acids or portions thereof are aligned in antiparallel orientation for maximum complementarity. According to some embodiments, if the window of evaluation is 15-16 nucleotides long, substantially complementary nucleic acids may have 0-3 mismatches within the window; if the window is 17 nucleotides long, substantially complementary nucleic acids may have 0-4 mismatches within the window; if the window is 18 nucleotides long, substantially complementary nucleic acids may have may contain 0-5 mismatches within the window; if the window is 19 nucleotides long, substantially complementary nucleic acids may contain 0-6 mismatches within the window. In certain embodiments the mismatches are not at continuous positions. In certain embodiments the window contains no stretch of mismatches longer than two nucleotides in length. In preferred embodiments a window of evaluation of 15-19 nucleotides contains 0-1 mismatch (preferably 0), and a window of evaluation of 20-29 nucleotides contains 0-2 mismatches (preferably 0-1, more preferably 0).

According to some embodiments, the RNAi molecules disclosed herein may be purified. Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, Mass.), or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

As used herein, the terms “single-stranded positive-strand virus” or “single-stranded positive-strand RNA virus”, refer to a virus having a genome of either “positive” (also referred to as “plus”) strand RNA i.e. protein is translated either directly from the viral genome or from RNA intermediaries having the same polarity as the corresponding viral mRNA.

As used herein, the term “SARS-CoV-2” is directed to a pleomorphic RNA virus of the Corona genus Coronavirus in the Coronaviridae. When infecting humans, the SARS-CoV-2 virus may result in the COVID-19 condition. The SARS-CoV-2 genome is represented by the nucleic acid sequence as set forth by Accession No.: NC_045512.2.

As used herein, the term viral “nucleoprotein” (also termed a “capsid protein” or a “nucleocapsid protein”) is a viral polypeptide that sequesters viral RNA and affects viral transcription. The viral nucleoprotein is capable of forming a nucleic acid/protein complex (i.e., a ribonucleoprotein (RNP) complex). A nucleoprotein is distinguished from an outer capsid protein, which generally does not contact and sequester the viral genome. The terms “nucleoprotein mRNA,” and “nucleoprotein transcript,” are understood to include any mRNA that encodes a viral nucleoprotein or its functional equivalent.

As used herein, the term “subject” includes humans and non-human mammals. In a preferred embodiment, the subject is a human. In other embodiments, the subject can be any other intermediate hosts capable of transmitting or transforming the virus into a human virus, such as but not limited to Rhinolophus sinicus. According to some embodiments, the subject is a patient, i.e. suffering from COVID-19 infections. According to some embodiments, the subject is a subject with an essentially asymptomatic COVID-19 infections. According to some embodiments, the subject is a subject suspected of being, yet not confirmed as being infected with SARS-CoV-2. According to some embodiments, the subject is a caregiver (e.g. a doctor, a nurse or family member) attending to a patient infected with or suspected as being infected with SARS-CoV-2.

As used herein, the term “host” may refer to any animal capable of being infected by the virus. According to some embodiments, the host is a human. According to some embodiments, the host is a mammal.

According to some embodiments, the subject may be an intermediate host. As used herein the term “intermediate hist” refers to a non-human mammal which poses high risk of transferring the virus to humans.

As used herein, the term “individual having high risk of severe illness if infected with SARS-CoV-2 may refer to older adults (e.g. above 60 or above 65), People who live in a nursing home or long-term care facility and people of any age who have serious underlying medical conditions might be at higher risk for severe illness from COVID-19, such as individuals suffering from moderate-to-severe asthma, chronic lung disease, diabetes, serious heart conditions, chronic kidney disease, severe obesity, immunocompromised subjects, or liver disease.

As used herein, the term “administration” to a subject can be carried out using known procedures, at dosages and for periods of time effective to provide the desired effect e.g. protection of the subject from infection, prevention of spread of the virus from an infected patient to a caregiver and/or for temporary reduction of viral load at least in the nasal and/or buccal tissues of the treated subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response.

Administration as used herein encompass both one subject providing administering the herein disclosed RNAi molecules or compositions comprising same to another subject as well as self-administration.

“Administering” includes routes of administration which allow the compositions of the invention to perform their intended function, e.g., proving protection from infection. A variety of routes of administration are possible including, but not necessarily limited to parenteral (e.g., intravenous, intraarterial, intramuscular, subcutaneous injection), oral (e.g., dietary), inhalation (e.g., aerosol to lung), topical, nasal, rectal, or via slow releasing microcarriers depending on the disease or condition to be treated. Inhalation and nasal and/or buccal spraying are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers.

In some embodiments, the composition is in a form suitable for inhalation. In some embodiments, the form of the composition is selected from the group consisting of nose drops, nasal sprays, sprayable liquid composition, inhalants and throat sprays. In some embodiments, the composition is provided in pressurized aerosol dosage form.

According to some embodiments, the RNAi molecules may be administered as naked RNA.

In some embodiments, there is provided a device comprising a pressurized aerosol dosage form of any one of the compositions disclosed herein, the device configured to produce aerosol from the pressurized aerosol dosage form. In some embodiments, the device is selected from the group consisting of nebulizer, MESH ultrasonic nebulizer, inhaler and atomizer. In some embodiments, said device is a hand-held device. Each possibility is a separate embodiment.

In some embodiments, said administering is via inhalation, e.g. utilizing apparatus configured to produce vapor and/or aerosol.

Inhalation administration can include an intranasal spray. Various forms suitable for administration by inhalation include aerosols, mists or powders. Each possibility is a separate embodiment.

Pharmaceutical compositions comprising the composition disclosed herein can be delivered in the form of an aerosol spray presentation from pressurized packaging or a nebulizer, e.g., with the use of a propellant (e.g., dichlorodifluoromethane, trichlorofluoromethane, dichlorotetrafluoroethane, carbon dioxide, or the like).

In some embodiments, the aerosol dosage form is encapsulated in a cartridge or a capsule.

In some embodiments, the composition disclosed herein is in a form of capsules or cartridges for use within a pressurized delivery system.

In some embodiments, the composition disclosed herein can be formulated into liquid compositions sprayable from non-aerosol packaging.

As used herein, the term “prevent infection” may refer to reduce the chance of a non-infected individual being infected with a virus e.g., SARS-CoV-2 when encountering an infected subject. For example, preventing infection of a caregiver attending to an infected subject.

As used herein, the term “reducing/attenuating/preventing spread” refers to reducing the chance of an infected subject infecting another subject during an encounter. As a non-limiting example, preventing spread may include preventing an infected patient from infecting others. As another non-limiting example, preventing spread may include preventing a caregiver being infected, optionally without knowledge thereof (e.g. due to the infection being asymptomatic or during latency) from spreading the infection to others.

As used herein, the term “reducing viral load” refers to, an optionally temporary reduction in the number of viral particles at least in treated tissues, such as nasal or buccal tissues.

As used herein, the term, a “pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

According to some embodiments, the transport vehicle may be a liposome. As used herein, the term “liposomes” refer to microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains. The decoy transcript may be completely or partially located in the interior space of the liposome, within the bilayer membrane of the liposome, or associated with the exterior surface of the liposome membrane. The liposome may facilitate or assist in the delivery of the decoy transcript into a target cell. The liposome may also protect the nucleic acid from an environment which may contain enzymes or chemicals that degrade nucleic acids and/or systems or receptors that cause the rapid excretion of the nucleic acids.

According to some embodiments, the transport vehicle may be a nanoparticle. As used herein, a “nanoparticle” refers to a colloidal particle for delivery of a molecule or agent that is microscopic in size of between or about between 1 and 1000 nanometers (nm), such as between 1 and 100 nm and behave as a whole unit in terms of transport and properties. Nanoparticles include those that are uniform in size. Nanoparticles include those that contain a targeting molecule attached to the outside.

According to some embodiments, the nanoparticle may be a lipid nanoparticle. As used herein, the term “lipid nanoparticle” refers to a transfer vehicle comprising one or more lipids (e.g., cationic lipids, non-cationic lipids, and PEG-modified lipids). Preferably, the lipid nanoparticles are formulated to deliver decoy transcript into target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

As used herein, the terms “approximately” or “about” in reference to a number are generally taken to include numbers that fall within a range of 5% or in the range of 1% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

As used herein, the singular forms “a,” “an” and “the” include plural referents unless the context clearly dictates otherwise.

As used herein, “optional” or “optionally” means that the subsequently described event or circumstance does or does not occur, and that the description includes instances where said event or circumstance occurs and instances where it does not.

According to some embodiments, there is provided an isolated ribonucleic acid interfering (RNAi) molecule, the RNAi molecule comprising a dsRNA sequence of from about 15 to about 60 base pairs or from about 15 to about 30 base pairs or from about 18 to about 24 base pairs, capable of decreasing expression of a SARS-CoV-2 gene.

According to some embodiments, the RNAi molecule comprises a nucleotide sequence having at least 70%, at least 80%, at least 85%, at least 90%, at least 95%, at least 98% or 100% sequence homology to an untranslated sequence (UTR) of SARS-CoV-2 (3′UTR or 5′ UTR).

According to some embodiments, the RNAi molecule comprises a nucleotide sequence having at least 70%, at least 80%, at least 85%, at least 90%, at least 95%, at least 98% or 100% sequence homology to the nsp8 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the nsp2 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the nsp3 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the nsp4 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the nsp6 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the nsp7 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the nsp9 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the nspll gene of SARS-CoV-2.

According to some embodiments, the RNAi molecule comprises a nucleotide sequence having at least 70%, at least 80%, at least 85%, at least 90%, at least 95%, at least 98% or 100% sequence homology to the 2′-0-ribose methyltransferase gene of SARS-CoV-2.

According to some embodiments, the RNAi molecule comprises a nucleotide sequence having at least 70%, at least 80%, at least 85%, at least 90%, at least 95%, at least 98% or 100% sequence homology to the 3′-To-5′ exonuclease gene of SARS-CoV-2.

According to some embodiments, the RNAi molecule comprises a nucleotide sequence having at least 70%, at least 80%, at least 85%, at least 90%, at least 95%, at least 98% or 100% sequence homology to the 3C-like proteinase gene of SARS-CoV-2.

According to some embodiments, the RNAi molecule comprises a nucleotide sequence having at least 70%, at least 80%, at least 85%, at least 90%, at least 95%, at least 98% or 100% sequence homology to the E-protein gene of SARS-CoV-2.

According to some embodiments, the RNAi molecule comprises a nucleotide sequence having at least 70%, at least 80%, at least 85%, at least 90%, at least 95%, at least 98% or 100% sequence homology to the endoRNAse gene of SARS-CoV-2.

According to some embodiments, the RNAi molecule comprises a nucleotide sequence having at least 70%, at least 80%, at least 85%, at least 90%, at least 95%, at least 98% or 100% sequence homology to the helicase gene of SARS-CoV-2.

According to some embodiments, the RNAi molecule comprises a nucleotide sequence having at least 70%, at least 80%, at least 85%, at least 90%, at least 95%, at least 98% or 100% sequence homology to a leader sequence of SARS-CoV-2.

According to some embodiments, the RNAi molecule comprises a nucleotide sequence having at least 70%, at least 80%, at least 85%, at least 90%, at least 95%, at least 98% or 100% sequence homology to the M-protein gene of SARS-CoV-2.

According to some embodiments, the RNAi molecule comprises a nucleotide sequence having at least 70%, at least 80%, at least 85%, at least 90%, at least 95%, at least 98% or 100% sequence homology to the N-protein gene of SARS-CoV-2.

According to some embodiments, the RNAi molecule comprises a nucleotide sequence having at least 70%, at least 80%, at least 85%, at least 90%, at least 95%, at least 98% or 100% sequence homology to the ORF lab gene of SARS-CoV-2.

According to some embodiments, the RNAi molecule comprises a nucleotide sequence having at least 70%, at least 80%, at least 85%, at least 90%, at least 95%, at least 98% or 100% sequence homology to the ORF10 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the ORF3a gene of SARS-CoV-2.

According to some embodiments, the RNAi molecule comprises a nucleotide sequence having at least 70%, at least 80%, at least 85%, at least 90%, at least 95%, at least 98% or 100% sequence homology to the ORF6 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the ORF7a gene of SARS-CoV-2.

According to some embodiments, the RNAi molecule comprises a nucleotide sequence having at least 70%, at least 80%, at least 85%, at least 90%, at least 95%, at least 98% or 100% sequence homology to the ORF7b gene of SARS-CoV-2.

According to some embodiments, the RNAi molecule comprises a nucleotide sequence having at least 70%, at least 80%, at least 85%, at least 90%, at least 95%, at least 98% or 100% sequence homology to the ORF8 gene of SARS-CoV-2.

According to some embodiments, the RNAi molecule comprises a nucleotide sequence having at least 70%, at least 80%, at least 85%, at least 90%, at least 95%, at least 98% or 100% sequence homology to the RNA-dependent RNA polymerase gene of SARS-CoV-2.

According to some embodiments, the RNAi molecule comprises a nucleotide sequence having at least 70%, at least 80%, at least 85%, at least 90%, at least 95%, at least 98% or 100% sequence homology to the S-protein gene of SARS-CoV-2.

According to some embodiments, the RNAi molecule comprises a nucleotide sequence having at least 70%, at least 80%, at least 85%, at least 90%, at least 95%, at least 98% or 100% sequence homology to any one of the SEQ ID NOs: 1-14425. According to some embodiments, the RNAi molecule comprises a nucleotide sequence having at least 70%, at least 80%, at least 85%, at least 90%, at least 95%, at least 98% or 100% sequence homology to any one of the SEQ ID NOs: 1-99; or SEQ ID Nos 1-80, or SEQ ID Nos: 3-10; or 4 and 6-9; or SEQ ID Nos: 11-29 and 82; or SEQ ID Nos: 12-13; or SEQ ID Nos: 30-46, 80 and 81; or SEQ ID Nos: 33-35 and 46; or SEQ ID Nos: 47-71 and 83-88; or SEQ ID Nos: 53, 54, 60, 62, 66, 68, 69, 83 and 84; or SEQ ID Nos: 72-78; or SEQ ID Nos: 76-78. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an untranslated sequence (UTR) of SARS-CoV-2 (3′UTR or 5′ UTR, the nucleotide sequence selected from the sequences set forth in SEQ ID NO: 1, 2 and 79, SEQ ID Nos 1 and 2. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to the RNA sequence encoding nsp8 of SARS-CoV-2, the nucleotide sequence selected from the sequences set forth in SEQ ID NOs: 3-10, set forth in SEQ ID NOs 4 and 6-9, or set forth in SEQ ID Nos. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an RNA sequence encoding helicase of SARS-CoV-2, the nucleotide sequence selected from the sequences set forth in SEQ ID NO: 11-29, 82 and 99; or set forth in SEQ ID Nos: 12-13. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an RNA sequence encoding the leader protein of SARS-CoV-2, the nucleotide sequence selected from the sequences set forth in SEQ ID Nos: 1, 2 and 79, set forth in SEQ ID NO: 1 and 2. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an RNA sequence being reverse complement to the leader protein of SARS-CoV-2, the nucleotide sequence selected from the sequences set forth in SEQ ID NOs: 72-78 or set forth in SEQ ID Nos: 76-78. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an RNA sequence encoding the RNA-dependent RNA polymerase of SARS-CoV-2, the nucleotide sequence selected from the sequences set forth in SEQ ID NOs: 30-46, 80 and 81 or set forth in SEQ ID Nos: 33-35 and 46. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an RNA sequence encoding the S-protein of SARS-CoV-2, the nucleotide sequence selected from the sequences set forth in SEQ ID NOs: 47-71 and 83-88 or set forth in SEQ ID Nos: 53, 54, 60, 62, 66, 68, 69, 83 and 84. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an RNA sequence encoding the nucleotide sequence selected from the sequences set forth in SEQ ID NOs: SEQ ID Nos 9353, 9452, 9549 and 14426-14428. Each possibility is a separate embodiment.

According to some embodiments, the RNAi molecule comprises a nucleotide sequence having at least 70%, at least 80%, at least 85%, at least 90%, at least 95%, at least 98% or 100% sequence homology to an RNA sequence encoding the nucleotide sequence set forth in SEQ ID NO: 9549.

According to some embodiments, the RNAi molecules may be stabilized. As used herein a “stabilized RNA” molecule may refer to RNA molecules that can contain stabilizing elements, including, but not limited to a 5′-cap structure or a 3′-poly(A) tail.

The 5′-capping of polynucleotides may be completed concomitantly during the in vitro-transcription reaction e.g. using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Each possibility is a separate embodiment. According to some embodiments, the capping comprises a 7-methylguanosine cap (m7G) or a m7G-analog.

According to some embodiments, the first and/or second strands of the RNAi molecule and the second strands have a 3′ single-stranded overhang. According to some embodiments, the overhang comprises at least 2, 3, 4, 5, 10 or more nucleotides. According to some embodiments, the overhang includes 2-10, 2-4 nucleotides. According to some embodiments, the overhang may be of exactly 2 nucleotides.

According to some embodiments, the RNAi molecule may be a siRNA or a shRNA. According to some embodiments, the RNAi molecule includes a sequence of at least 15 contiguous nucleotides that are complementary or substantially complementary to a sequence within the genome of the SARS-CoV2 genome. According to some embodiments, the RNAi molecule may be a siRNA or a shRNA. According to some embodiments, the RNAi molecule includes a sequence of at least 15 contiguous nucleotides that are complementary or substantially complementary to a opposite strand sequence of the SARS-CoV-2 genome.

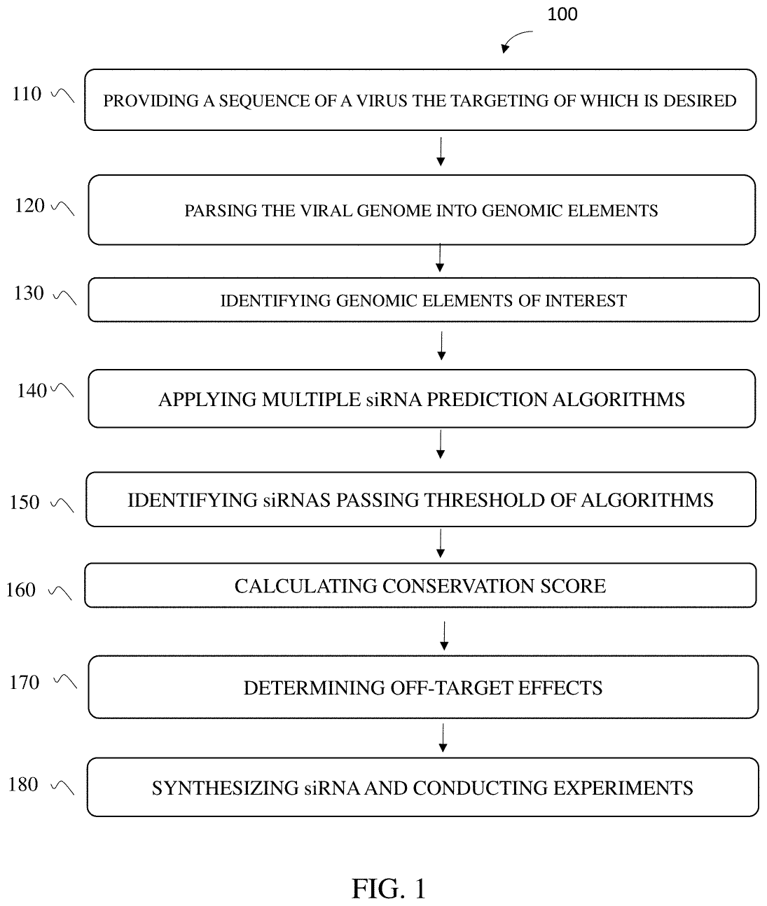

Reference is now made to FIG. 1 which is a flowchart of an optional method 100 for identifying potential RNAi molecules.

According to some embodiments, a list of siRNA molecules complementary to SARS-CoV-2 may be provided using the below listed guidelines and set forth in FIG. 1. According to some embodiments, the siRNA may have a length of 19nt. Advantageously, such rather short RNAi molecules reduce the cost of the final therapeutic product while still ensuring target specificity.

In step 110 of the method, a sequence of a virus the targeting of which is desired is provided, for example, using a BioPython function that downloads a NCBI entry of the virus.

In step 120 the viral genome is parsed into its genomic elements, e.g. based on the information from the NCBI entry. For each element, a distinct file that describes the underlying genomic sequence is generated. Optionally, the element may be chopped into d pieces, where d may be a user specified value.

In step 130, genomic elements of interest are identified.

In step 140, multiple siRNA prediction algorithms that assess the genomic elements are applied. Non-limiting examples of suitable algorithms include: RNAxs (http://rna.tbi.univie.ac.at/cgi-bin/RNAxs/RNAxs.cgi), DSIR (http://biodev.cea.fr/DSIR/DSIR.php), and OligoWalk (http://rna.urmc.rochester.edu/cgi-bin/server_exe/oligowalk/oligowalk.pl), and for each genomic element, the output of all prediction algorithms is harmonized.

In step 150, siRNA molecules that pass a predetermined selection threshold in more than one algorithm are identified. This increases the chance that the siRNA is indeed potent and has high efficacy. Optionally, a cross-correlation matrix is calculated. Without being limited by any theory, a loose correlation, suggests that each algorithm captures unique information.

In step 160, a conservation score is calculated for each siRNA that passed all prediction algorithms. To this end, all known viral strains of the target virus are first identified. The sequence of each strain is then scanned and overlapping k-mer sequences extracted, where k=19nt to match the siRNA target size. A hash data structure that maps the k-mer sequence to all strains that it appeared in is then recorded from extracted k-mers. The hash data structure is then iterated to enumerate the number of strains that the specific 19nt target sequence appears in, the number being serving as the conservation score. It is understood that while conserved sequences are preferred, this step is optional.

In step 170, the possibility of off-target effects is evaluated. Optionally two types of algorithms are utilized:
a. The melting temperature of the seed region of the guide and the passenger RNA strands of the siRNA are calculated. The melting temperature is calculated using the nearest-neighbor method number 1 of BioPython with no slat correction. Advantageously, this setting obtains the closest melting predictions as in Ui-Tei et al., Nucleic Acid Research, 2008 with a Pearson correlation of over 85%.
b. The number of matches of the siRNA target to potential host transcripts is calculated. Previous studies have suggested aligning the siRNA strands to the human genome. However, since most of the genome is not transcribed and since many transcripts are the product of splicing, this approach was declined. Instead, the human transcriptome is downloaded (Homo_sapiens.GRCh38.cdna.all) e.g. from the Ensembl website and transformed to a Burrows-Wheeler (BWT) data structure using Bowtie v1. Each siRNA was then aligned to the human transcriptome in a BWT structure using bowtie. Specifically, the following parameters ‘-n3-18-p4-a-y’ were used so as to increase the sensitivity of Bowite to short targets, such as siRNA targets. Two numbers were calculated: 1) the number of human transcripts that contain a close match to the guide or passenger siRNA strands and 2) the number of transcripts that contain a loose match to the guide or passenger targets. A close match is defined as zero mismatches between the seed regions of either strands to the transcript or up to one match in the rest of the siRNA. A loose match is defined as up to one mismatch in the seed region or up to two mismatches in the entire siRNA.

In step 180, the siRNAs are synthesized for experimental evaluation.

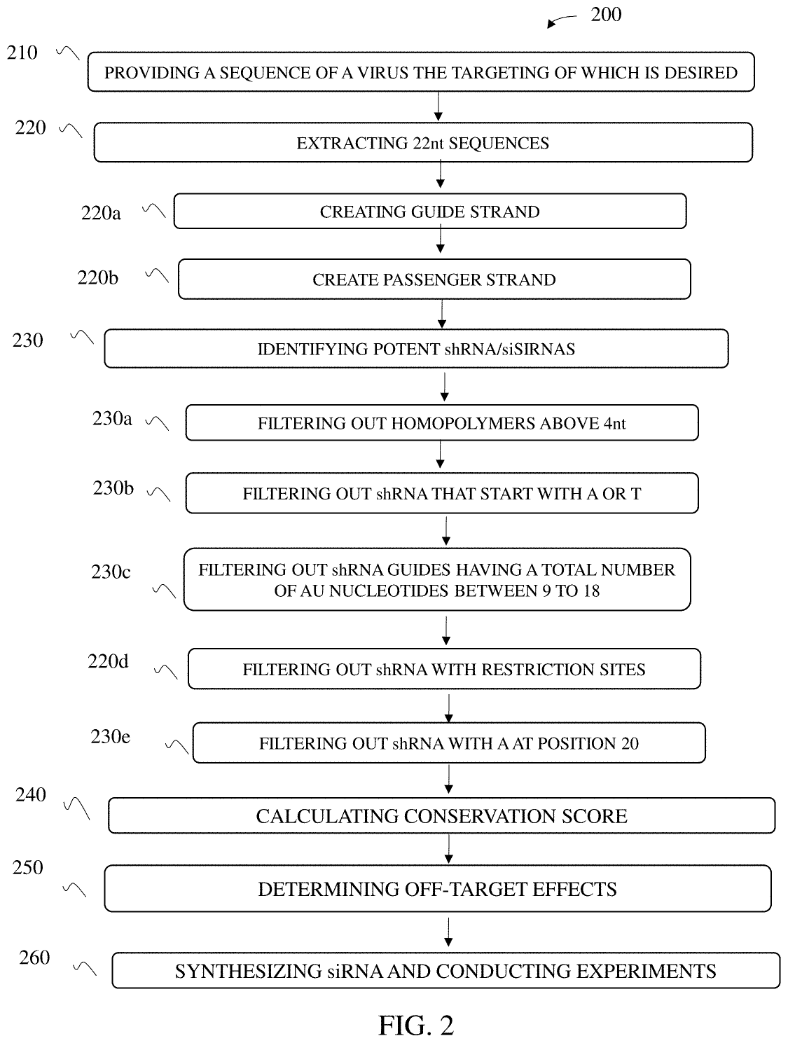

Reference is now made to FIG. 2, which is a flowchart of an additional or alternative optional method 200 for identifying potential RNAi molecules.

According to some embodiments, a list of siRNA molecules complementary to SARS-CoV-2 may be provided using the below listed guidelines and set forth in FIG. 2. According to some embodiments, the RNAi molecules (shRNA or siRNA).

In step 210 of the method, a sequence of a virus the targeting of which is desired is provided, for example, using a BioPython function that downloads a NCBI entry of the virus.

In step 220, the viral genome is scanned and strings of 22nt sequences are extracted from it with shifts of one nucleotide. Each 22mer is a potential target region and undergoes the following sub steps:
a. The 22nt sequence of the target region is reversed and complemented and this is the guide strand of the shRNA or the siRNA.
b. If shRNA is created, the guide strand is reversed and complemented one more time to create the passenger strand. The passenger strand is subject to another processing step such that if the 3′ end of the guide strand is either A or T, then the 5′ end of the passenger strand is set to “C”. Alternatively, if the 3′ end of the guide strand is either C or G, then the 5′ end of the passenger strand is set to “A”.
c. If siRNA is created, the guide strand is reversed and complemented one more time to create the passenger strand. Both the guide and the target are transcribed, and optionally a “UU” overhang at the 3′ end of each of the strands is added.

In step 230, potentially potent shRNA/siRNAs are identified by subjecting the guide and the passenger strands to one or more (optionally all) of the following steps:
a. shRNAs whose targets (plus 14nt from each side) have homopolymers above 4nt are filtered out. This step may be taken since such homopolymers are hard to synthesize and therefore are less likely to create an effective clinical grade shRNA/siRNA molecule. In addition, homopolymers tend to be genomically unstable and undergo rapid evolution, which help the virus to escape the RNAi treatment.
b. shRNA guides that start with either A or T are filtered out. Without being bound by any theory, this filtering significantly increases the odds of an shRNA to be highly potent.
c. shRNA guides having a total number of AU nucleotides between 9 to 18 are filtered out. Without being bound by any theory, this filtering significantly increases the odds of an shRNA to be highly potent.
d. shRNAs containing certain restriction sites are filtered out. Without being bound by any theory, this enables better cloning in case shRNA rather than siRNA molecules are used as the therapeutic agent.
e. Optionally, shRNA guides that have no “A” in position 20 are identified. Without being bound by any theory, this filtering significantly increases the odds of an shRNA to be highly potent.

In step 240, a conservation score is calculated, as essentially described above. However, instead of k=19nt, a k=22nt was applied in order to be compatible with the longer guide and passenger strands. It is understood that while conserved sequences are preferred, this step is optional.

In step 250, the possibility of off-target effects are evaluated as essentially described above with the modification that any RNAi molecules having passenger or guide strands with less than 2 mismatches or less than 1 mismatch to a human transcript in the seed region were filtered out.

In step 260, the siRNAs are synthesized for experimental evaluation.

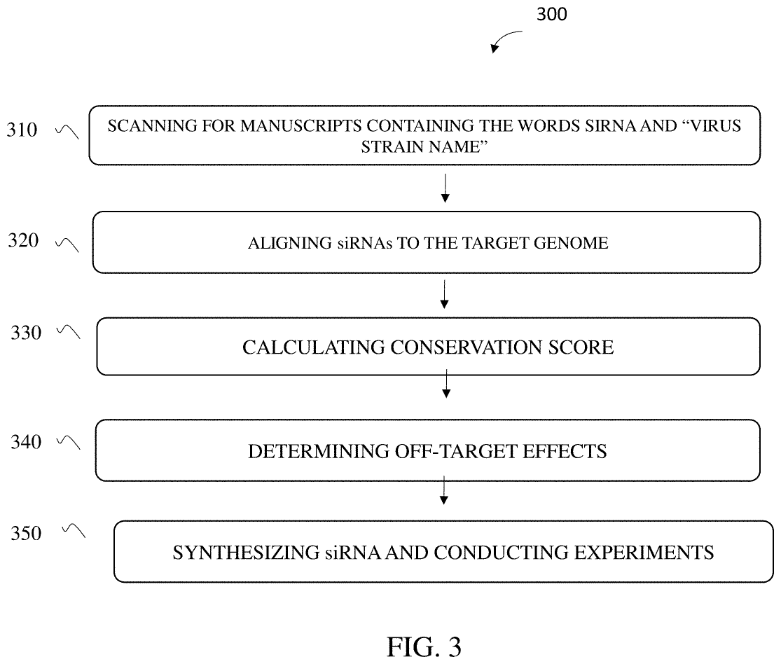

Reference is now made to FIG. 3, which is a flowchart of an additional or alternative optional method 300 for identifying potential RNAi molecules.

According to some embodiments, a list of siRNA molecules complementary to SARS-CoV-2 may be provided using the below listed guidelines and set forth in FIG. 3. According to some embodiments, the RNAi molecules (shRNA or siRNA).

This method is based on repurposing siRNAs that have been identified as potent in strains similar to the target virus of interest. A notable challenge of this approach is when the sequence homology is of 70% or less., Without being bound by any theory, the probability of finding stretches of identical 19nt nucleotides may be roughly estimated to be in the order of of 0.7\hat{ }19=0.0013. On the other hand, if identified such siRNA likely reflect evolutionary conserved regions and therefore are likely to offer protection against a wide variety of strains.

In step 310 of the method, NCBI's PubMed manuscripts are scanned for the words siRNA and “virus strain name”. The text is then scanned to find uninterrupted strings of letters that are 19 to 25 letters long without a gap and that contain only the following letters: A, C, G, T or U. In addition, in some embodiments, the virsiRNAdb database is scanned for entries for the virus name and these entries are downloaded and the siRNA sequences are parsed.

In step 320, the identified siRNAs are aligned to the target genome and to its reverse complement without allowing for gaps or mismatches besides at the 3′ tail that sometimes have an overhang.

In steps 330 and 340 conservation scores and off-target effects are determined as described with respect to FIG. 1 and the RNAi molecule synthesized (step 350).

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

Example 1—Identifying siRNA Molecules Against the SARS-CoV-2

A list of siRNA molecules complementary to SARS-CoV-2 was provided using the method as essentially outlined in FIG. 1.

In short, the BioPython function was utilized to download the entry “NC_045512.2” (set forth in SEQ ID NO: 1) from the NCBI website. This entry describes the reference genome of the SARS-CoV-2 from a Wuhan patient and is given in an annotated format that indicates the different genomic elements of the virus.

The genome SARS-CoV-2 was then parsed into its various genomic elements based on the information from the NCBI entry. For each element, a distinct file that describes the underlying genomic sequence was generated. Element having a length of above 10000nt were chopped into 3 pieces.

The following genomic elements were identified as being of interest: 5′UTR, nsp8 (primase), nsp12 (RNA-dependent RNA polymerase), 3′UTR, Spike, and the entire ORFlab.

Next, multiple siRNA prediction algorithms, that assess the genomic elements, were applied. Specifically, RNAxs (http://rna.tbi.univie.ac.at/cgi-bin/RNAxs/RNAxs.cgi), DSIR (http://biodev.cea.fr/DSIR/DSIR.php), and OligoWalk (http://rna.urmc.rochester.edu/cgi-bin/server_exe/oligowalk/oligowalk.pl) were utilized.

For each genomic element, the output of all prediction algorithms was harmonized and siRNA molecules that pass a predetermined selection threshold in all algorithms were identified.

For the identified siRNA molecules, a conservation score was calculated. To this end, all known viral strains of SARS-CoV2 available at the NCBI website were identified. The sequence of each strain were scanned and overlapping 19nt sequences were extracted, based upon which the conservation score was calculated.

Next, for the conserved siRNA, the possibility of off-target effects was evaluated using two types of algorithms as described above:
a. The melting temperature of the seed region of the guide and the passenger RNA strands of the siRNA was calculated.
b. The number of matches of the siRNA target to potential human transcripts was calculated.

522 siRNAs were identified and 80 synthesized for experimental evaluation.

### Example 2—Identifying siRNA Molecules Against the SARS-CoV-2

A list of siRNA molecules complementary to SARS-CoV-2 was provided using the method as essentially outlined in FIG. 2.

This pipeline generates a list of shRNA/siRNA molecules of 22nt that are reactive against SARS-CoV-2.

In short, the BioPython function was utilized to download the entry “NC_045512.2” (set forth in SEQ ID NO: 1) from the NCBI website. This entry describes the reference genome of the SARS-CoV-2 from a Wuhan patient and is given in an annotated format that indicates the different genomic elements of the virus.

Next, the SARS-CoV-2 reference genome was scanned and 22nt substrings with shifts of one nucleotide were extracted. For each 22mer the following sub steps were carried out:
a. The 22nt sequence of the target region was reversed and complemented to provide the guide strand of the shRNA.
b. The guide strand was then reversed and complemented one more time to create the passenger strand. The passenger strand was subsequently subject to another processing step such that if the 3′ end of the guide strand is either A or T, the 5′ end of the passenger strand is set to “C” and if the 3′ end of the guide strand is either C or G, then the 5′ end of the passenger strand is set to “A”.

Following the filtering described in step 230a-230d of FIG. 2, off-target effects were determined and suitable siRNAs and shRNA synthesized.

In total, 14425shRNA molecules were identified.

### Example 3—Identifying siRNA Molecules Against the SARS-CoV-2

A list of siRNA molecules complementary to SARS-CoV-2 was provided using the method as essentially outlined in FIG. 3.

In short, the NCBI's PubMed databases was search for manuscripts with the words siRNA and SARS and the text scanned to find uninterrupted strings of letters that are 19 to 25 letters long without a gap and contain only the following letters: A, C, G, T or U.

The identified siRNA entries were then aligned to the SARS-CoV-2 genome without allowing for gaps or mismatches besides at the 3′ tail that sometimes have an overhang. From 9422 sequences extracted from the PMCID literature, only 31 aligned to the SARS-CoV-2 genome.

### Example 4—Validation of siRNA Efficiency—Reporter Assay

The identified RNAi molecules are inserted into an expression vector including a reporter, such as mCherry. The insertion is such that if the RNAi molecule is effective against its target, expression of the reporter (mCherry) decreases.

In short, HEK 293 FT cells were transfected in duplicate with a) a viral genomic region of interest (e.g., Helicase, Polymerase, Spike, Nsp8, 3′ UTR, Leader or Leader reverse complement transcripts) fused to a reporter gene (e.g., Cherry); b) a reporter gene (e.g., GFP) for normalization of transfection efficiency; and c) the siRNA to be tested.

The total plasmid amount was 500 ng (equimolar ratio), and the siRNA concentration 1 nM (i.e., significantly lower than commercial concentrations of siRNAs which is around 10-30 nM). Anti-GFPsiRNA served as a positive control (at 30 nM).

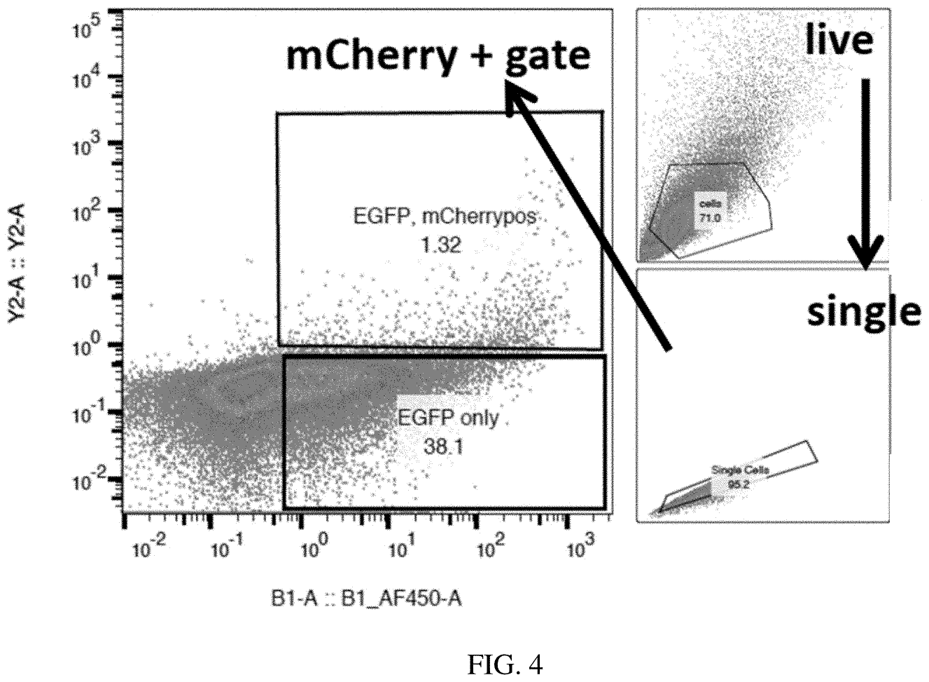

The cells were analyzed by FACS as follows (FIG. 4):
1. Initially live cells were gated using FSC vs SSC channels—upper right panel;
2. Second single cells were gated (FSC-A vs FSC-H)—lower right panel;
3. GFP positive cells (indicative of transfected cells) were then identified; and
4. Lastly, the fractions of mCherry+/GFP+ vs. GFP+ only cells counted, indicative of inefficient and efficient siRNA constructs respectively (left panel).

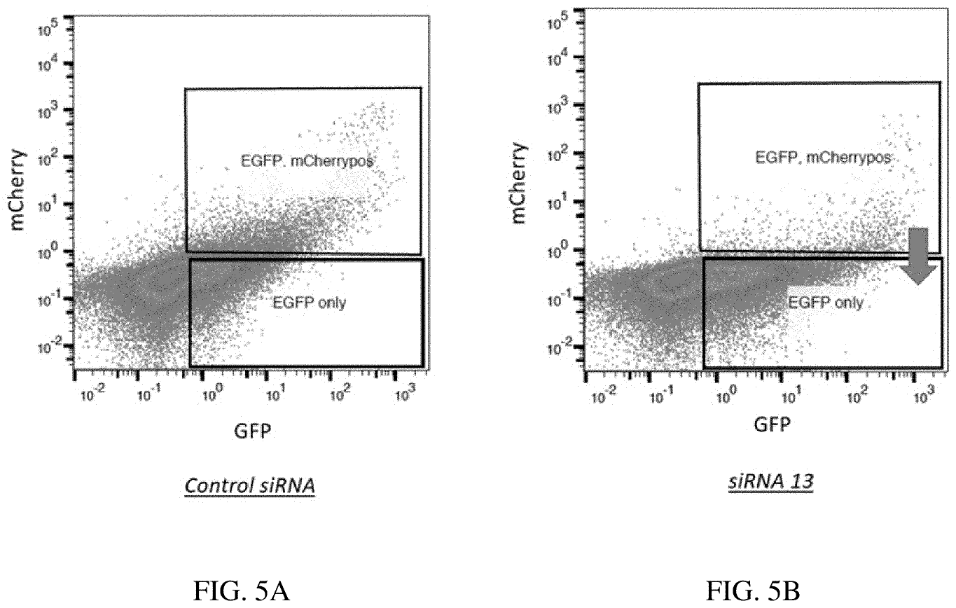

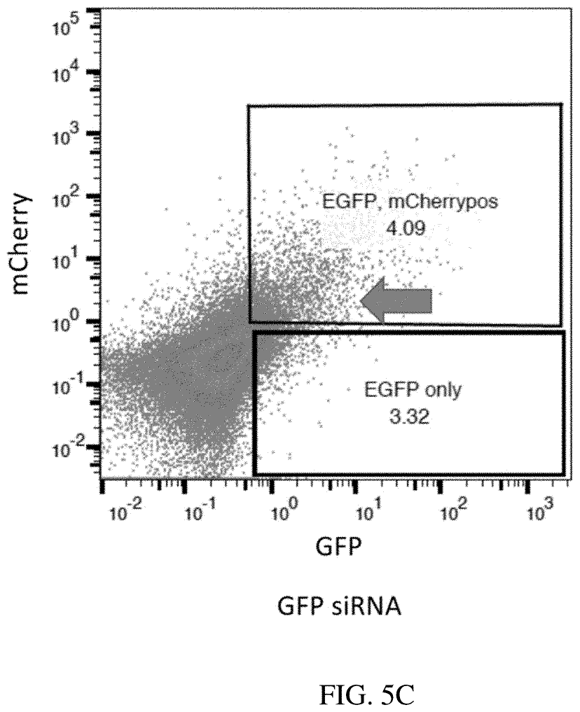

Representative FACS results of cells transfected with a control siRNA (negative control), target siRNA and siRNA against GFP (positive control) are shown in FIG. 5A, FIG. 5B and FIG. 5C respectively.

Specifically, the following siRNA sequences were tested three different concentrations of siRNA (1 nM, 500 pM and 100 pM):

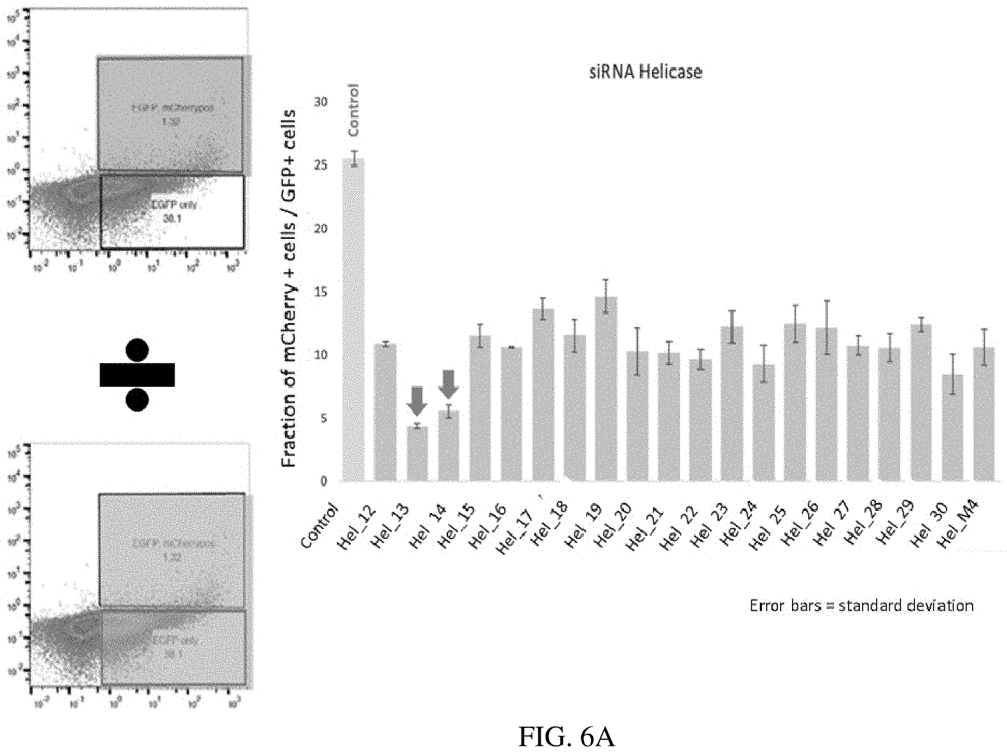

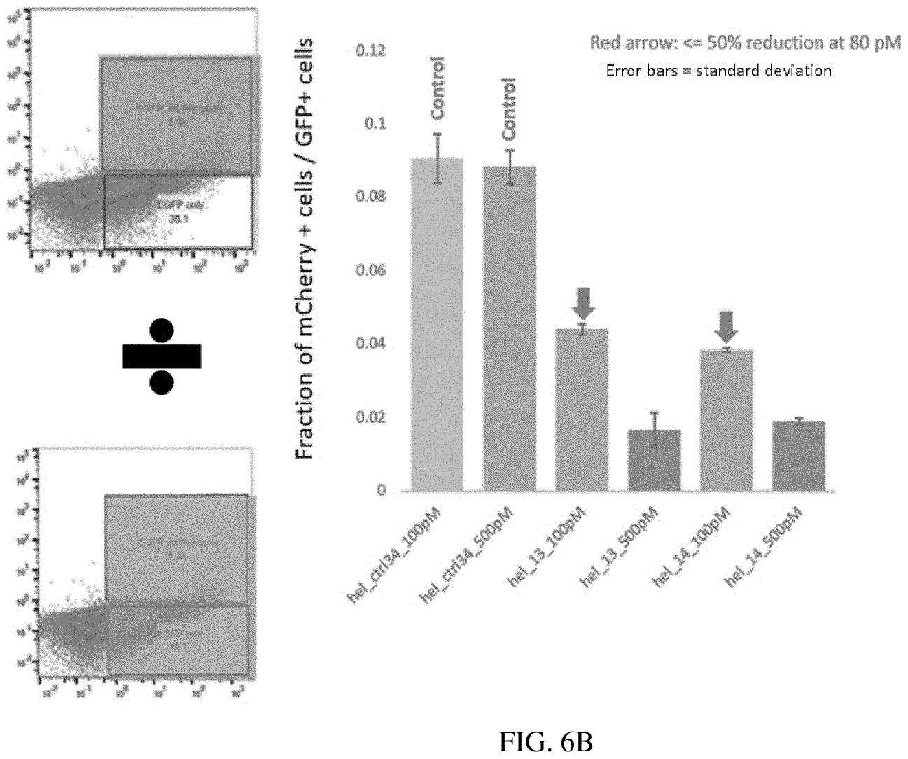

20 siRNAs against the helicase of SARS-CoV-2 were tested (SEQ ID NOs 11-29 and 82). As seen from FIG. 6A and all the tested siRNA sequences caused a significant reduction in mCherry+GFP/GFP+ ratio indicative of them being efficient siRNAs. A particularly impressive reduction in the ratio was observed for siRNAs hel_13 (SEQ ID NO: 12) and hel_14 (SEQ ID NO: 13)—indicated by arrows. As further seen from FIG. 6B, siRNAs hel_13 and hel_14 caused a 50% or larger reduction in the mCherry+GFP/GFP+ ratio at 80 pM concentration, indicating them being highly potent siRNAs.

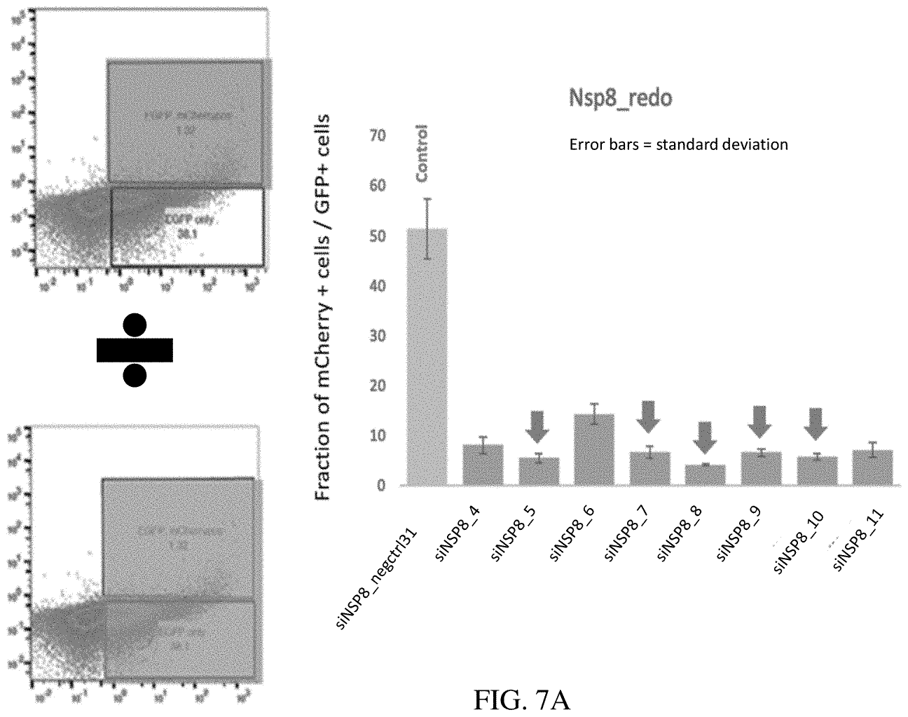

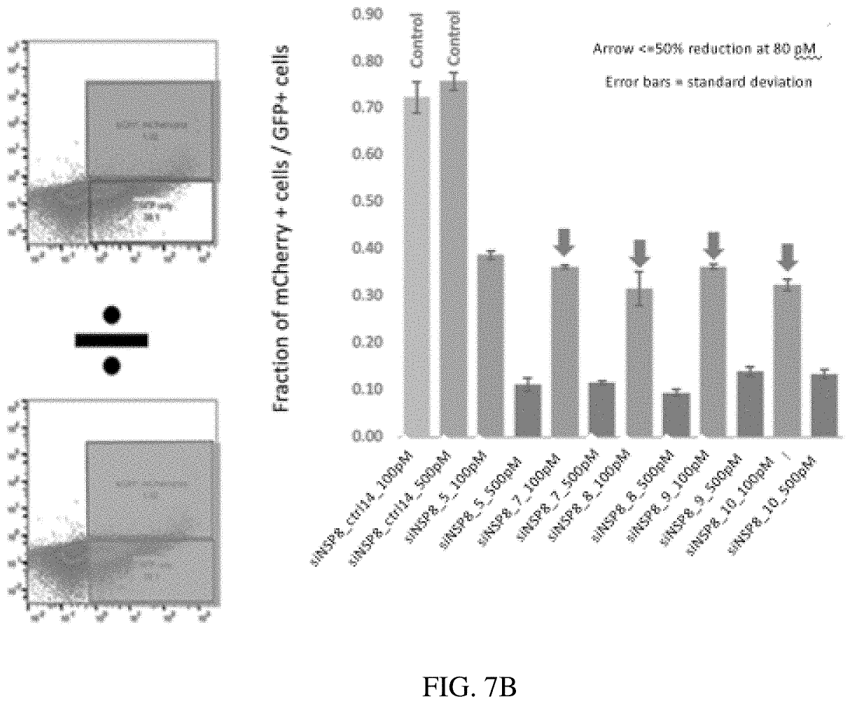

8 siRNAs (SEQ ID Nos: 3-10) against Nsp8 of SARS-CoV-2 were tested. As seen from FIG. 7A, all the tested siRNA sequences caused a significant reduction in mCherry+GFP/GFP+ ratio indicative of them being efficient siRNAs. A particularly impressive reduction in the ratio was observed for siRNAs siNSP8_5 (SEQ ID NO: 4), siNSP8_7 (SEQ ID NO: 6), siNSP8_8 (SEQ ID NO: 7), siNSP8_9 (SEQ ID NO: 8) and siNSP8_10 (SEQ ID NO: 9)—indicated by arrows). As further seen from FIG. 7B, siRNAs siNSP8_7, siNSP8_8, siNSP8_9 and siNSP8_10 caused a 50% or larger reduction in the mCherry+GFP/GFP+ ratio at a 80 pM concentration, indicating them being highly potent siRNAs.

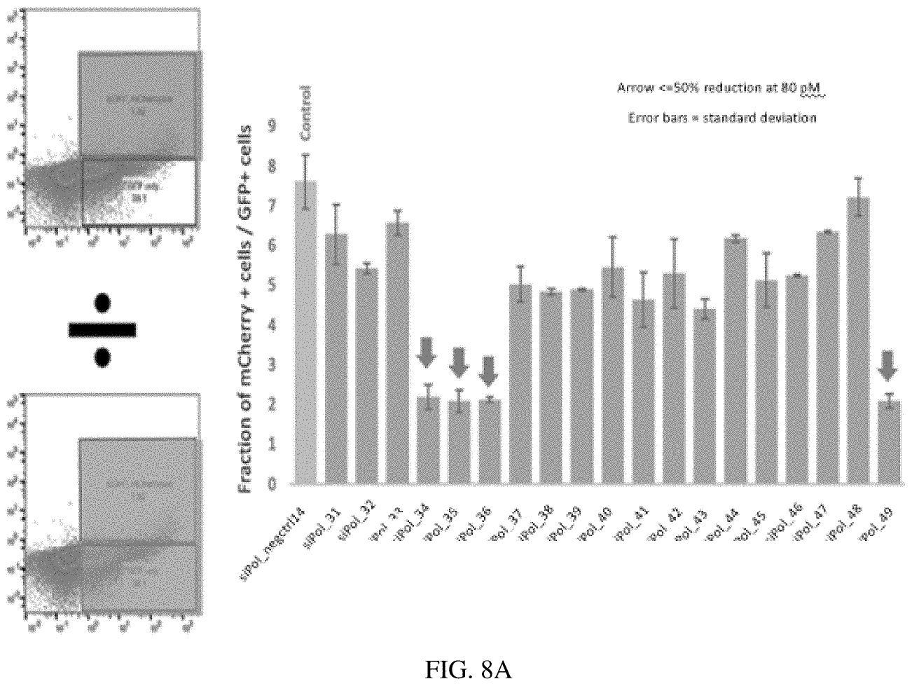

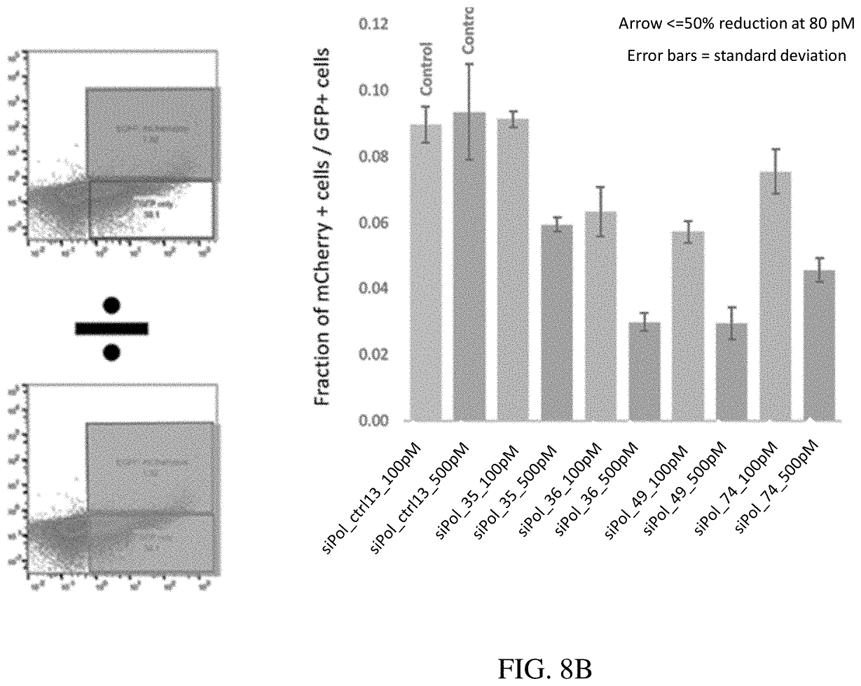

19 siRNAs (SEQ ID Nos: 30-46, 80 and 81) against the RNA dependent RNA polymerase of SARS-CoV-2 were tested. As seen from FIG. 8A, all the tested siRNA sequences caused a reduction in mCherry+GFP/GFP+ ratio indicative of them being efficient siRNAs. A particularly impressive reduction in the ratio was observed for siRNAs siPol_34 (SEQ ID NO: 33), siPol_35 (SEQ ID NO: 34), siPol_36 (SEQ ID NO: 35) and siPol_49 (SEQ ID NO: 46)—indicated by arrows. FIG. 8B, siRNAs siPol_36 and siPol_49 caused an almost 50% reduction in the mCherry+GFP/GFP+ ratio at a 80 pM concentration, indicating them being highly potent siRNAs.

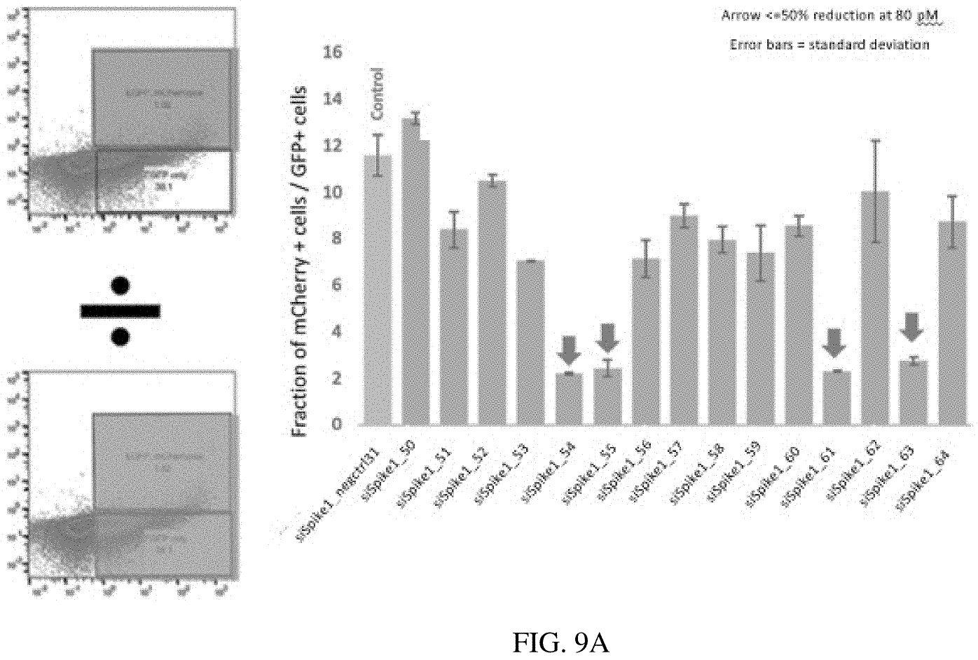

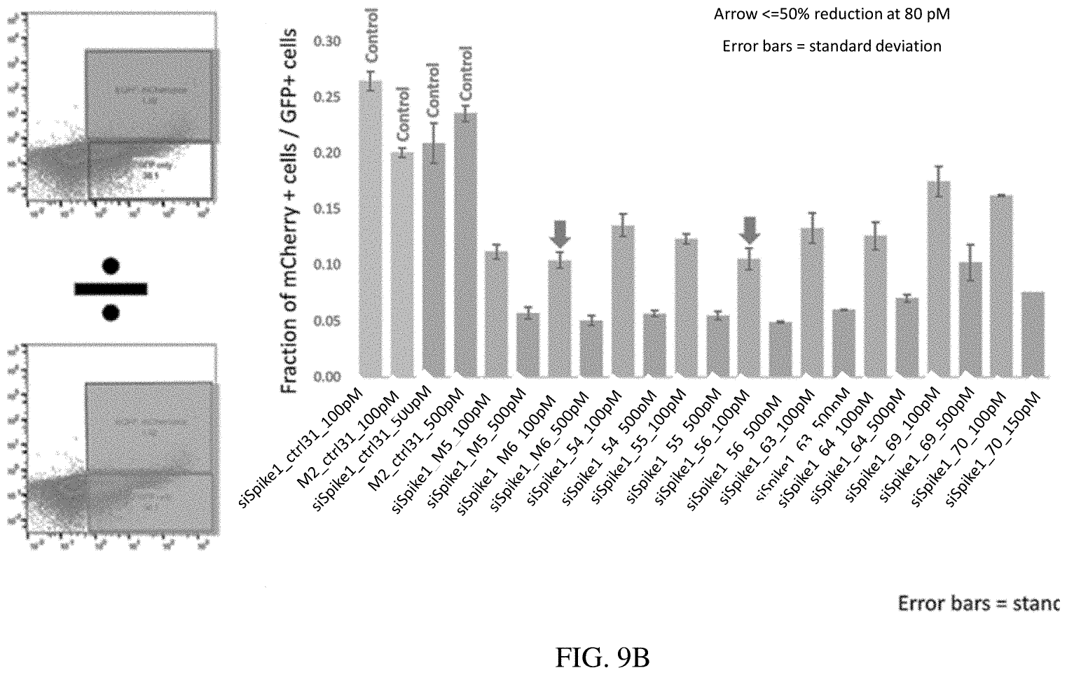

15 siRNAs (SEQ ID Nos: 50-64) against Spike1 of SARS-CoV-2 were tested. As seen from FIG. 9A, all but one of the tested siRNA sequences caused a reduction in mCherry+GFP/GFP+ ratio indicative of them being efficient siRNAs. A particularly impressive reduction in the ratio was observed for siRNAs siSpike1_54 (SEQ ID NO: 53), siSpike1_55 (SEQ ID NO: 54), siSpike1_61 (SEQ ID NO: 60) and siSpike1_63 (SEQ ID NO: 62)—indicated by arrows. As further seen from FIG. 9B, siRNA siSpike1_61 caused a larger than 50% reduction in the mCherry+GFP/GFP+ ratio at a 80 pM concentration, indicating them being highly potent siRNAs.

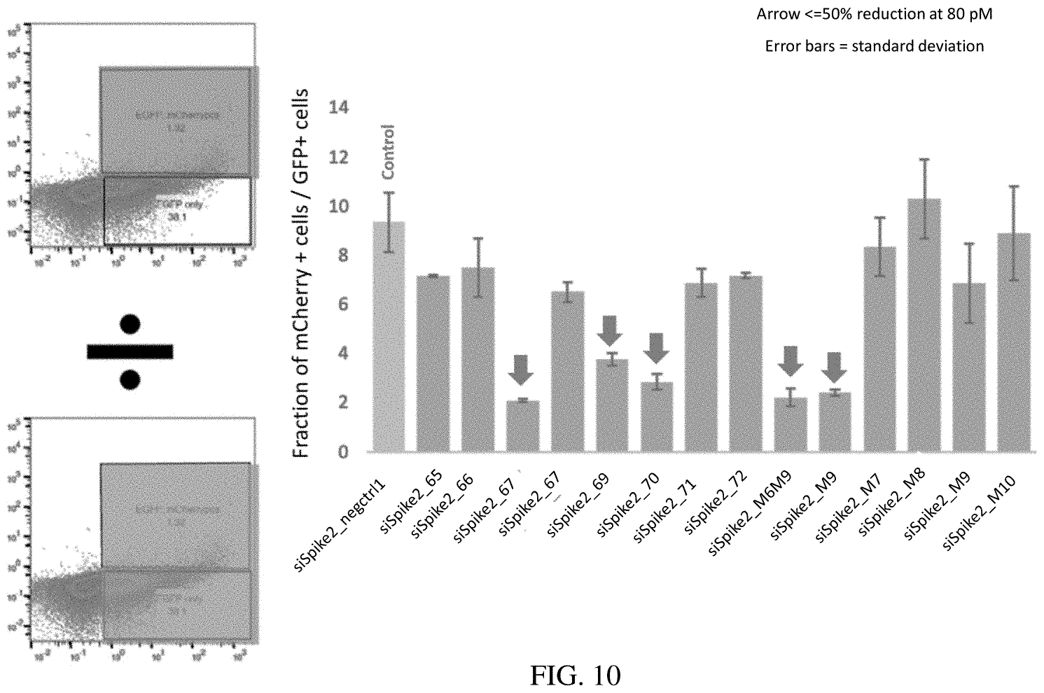

14 siRNAs (SEQ ID NOs: 64-71 and 83-88) against Spike2 of SARS-CoV-2 were tested. As seen from FIG. 10, all but two of the tested siRNA sequences caused a reduction in mCherry+GFP/GFP+ ratio indicative of them being efficient siRNAs. A particularly impressive reduction in the ratio was observed for siRNAs siSpike2-67 (SEQ ID NO: 66), siSpike2-69 (SEQ ID NO: 68), siSpike2_70 (SEQ ID NO: 69), siSpike2_M6M9 (SEQ ID NO: 84) and siSpike2_M5 (SEQ ID NO: 83)—indicated by arrows. As further seen from FIG. 9B, siRNA siSpike2_M6 (SEQ ID NO: 84) caused a larger than 50% reduction in the mCherry+GFP/GFP+ ratio at a 80 pM concentration, indicating them being highly potent siRNAs.

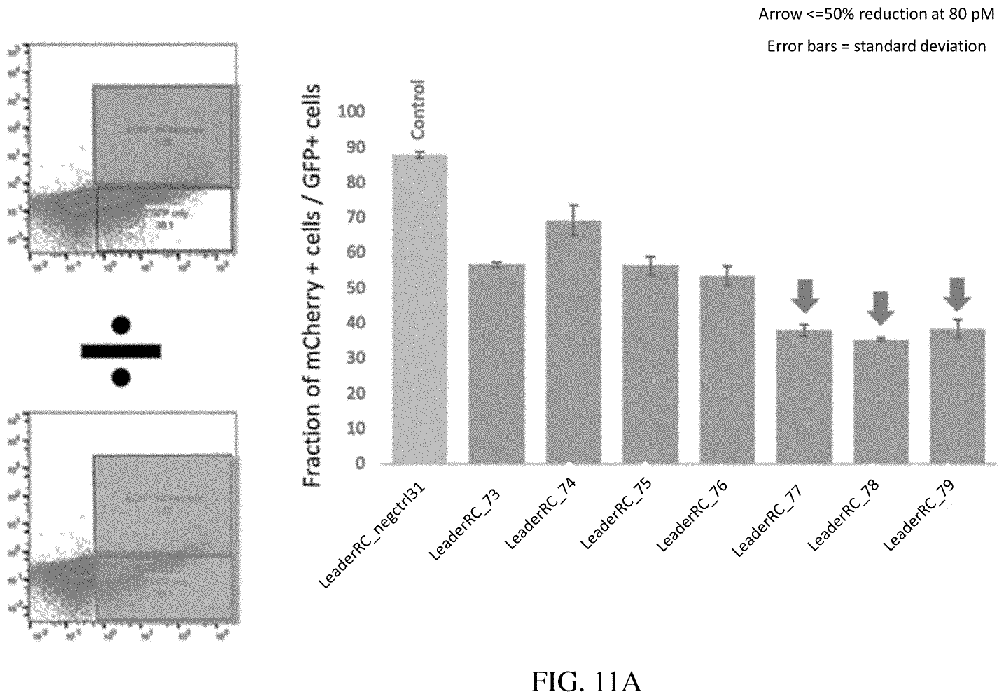

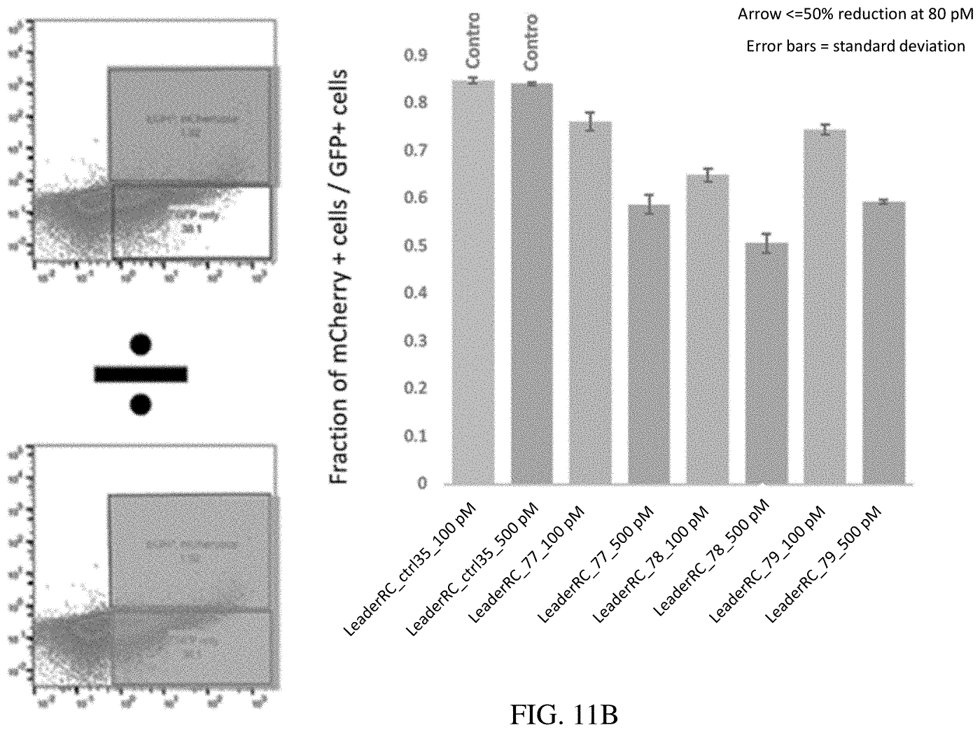

7 siRNAs (SEQ ID NOs: 72-81) targeting the reverse complement of the Leader of SARS-CoV-2 were tested. As seen from FIG. 11A, all the tested siRNA sequences caused a reduction in mCherry+GFP/GFP+ ratio indicative of them being efficient siRNAs. A particularly impressive reduction in the ratio was observed for siRNAs leaderRC 77 (SEQ ID NO: 76), leaderRC_78 (SEQ ID NO: 77) and leaderRC 79 (SEQ ID NO: 78)—indicated by arrows. As further seen from FIG. 11B, lower concentrations of siRNA where less efficient for the tested siRNAs, but a close to 50% reduction in the mCherry+GFP/GFP+ ratio was observed for leaderRC_78 at a 500 pM concentration.

### Example 5—Validation of siRNA Efficiency—Reporter Assay in Stably Transfected Cell Lines

The reporter assay was repeated in a stably transfected cell line which enables to reliably measure fluorescence intensity and not just number of fluorescent cells and thus provide a quantitative indication of siRNA efficiency.

4 NSP8 siRNAs were tested (NSP8_7 (SEQ ID NO: 6), NSP8_8 (SEQ ID NO: 7), NSP8_10 (SEQ ID NO: 9)) and NSP_14 (SEQ ID NO: 13) and compared to a control siRNA.

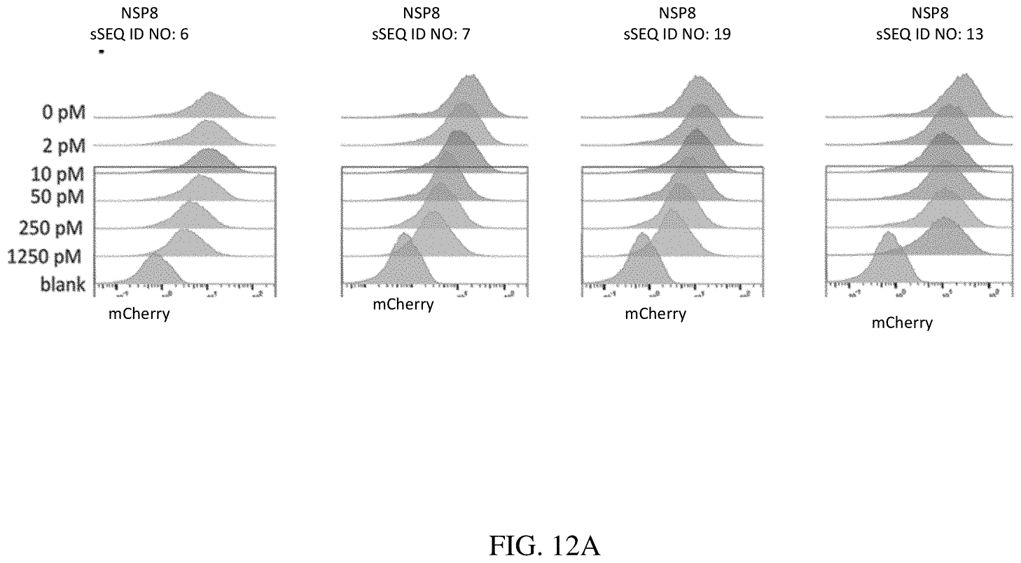

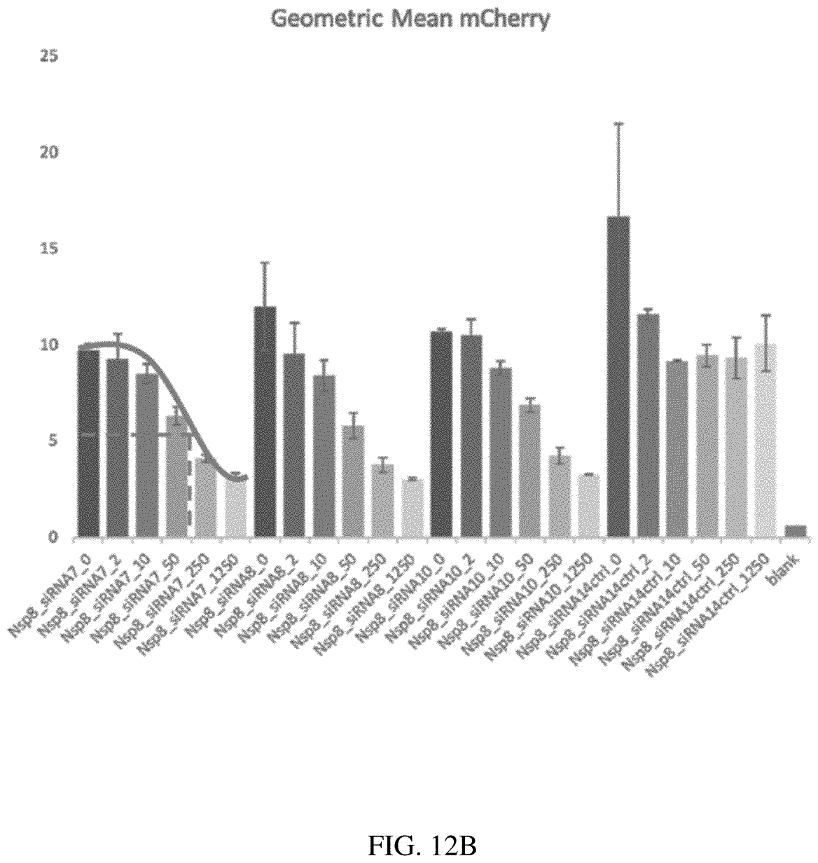

As seen from FIG. 12A and FIG. 12B, an IC50 of fluorescence was observed at a concentration of between about 50 pM and 100 pM for all four siRNAs, indicating the high efficiency of these siRNAs.

10 additional siRNAs siRNAs targeting various regions of the SARS-COV2 genome were also tested (S1-S10) and compared to a control siRNA.

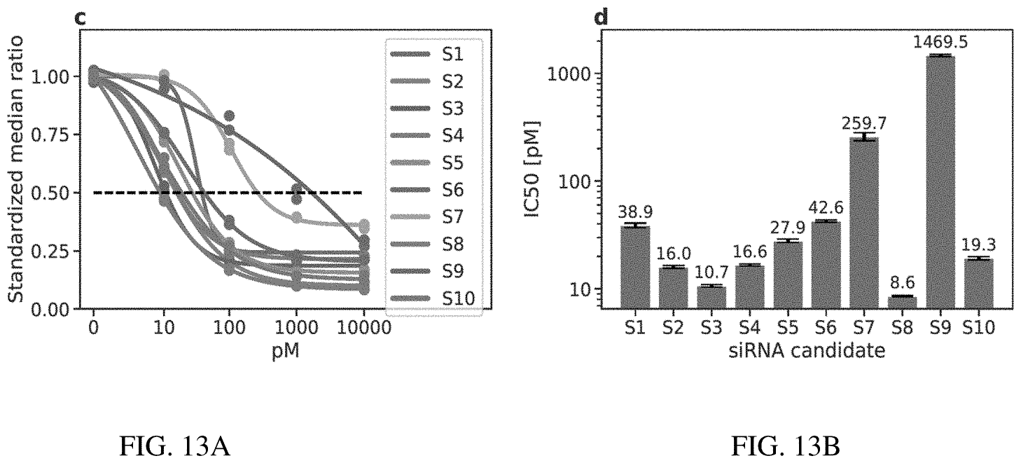

As seen from FIG. 13A and FIG. 13B, an IC50 of fluorescence was observed at a concentration of below 20 pM was observed for 5 of the siRNAs, namely S2, S3, S4, S8 and S10 indicating the high efficiency of these siRNAs.

### Example 6—Validation of siRNA Efficiency—Viral Protection qPCR Assay

Vero E6 cells (known to be easily infected by Sars-COV2) were transfected with increasing concentration of candidate siRNAs targeting various part of the of SARS-COV-2 genome, namely 51, S2, S3 and S5-S9, set forth in SEQ ID Nos: 89-91 and 93-97 (100 nM) and challenged 24h after with 600 TCID50 (¬0.1 MOI) of Sars-COV2.

48 hours post-infection viral load was determined by qPCR Allplex™ SARS-CoV-2 Assay by Seegene using standard protocol. The subgenomic region is an average of the RNA levels of the “N” and “E” gene and the genomic region reflects the RNA levels of Orf1_ab.

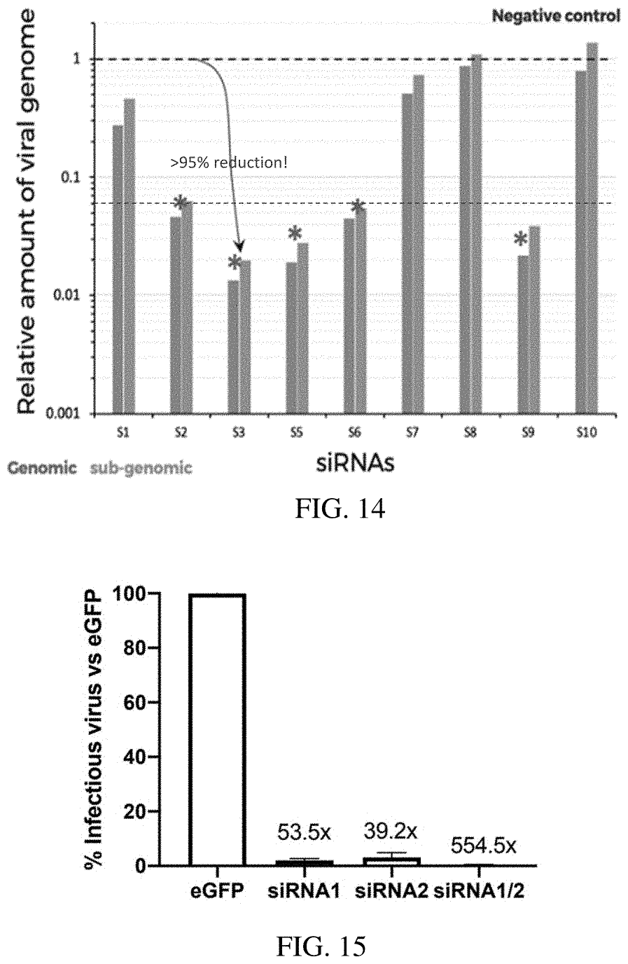

As seen from FIG. 14, several siRNAs targeting SARS-CoV-2 genome caused a larger than 95% reduction in viral load namely siRNAs S2, S3, S5, S6 and S9, set forth in SEQ ID NOs: 90, 91, 93, 94 and 97, indicating the impressive ability of these siRNAs to attenuate live SARS-COV2 viruses.

Similarly, Vero E6 cells (known to be easily infected by SARS-COV2) were transfected with siRNAs targeting NSP8 and RNA dependent RNA polymerase (SEQ ID NO: 8 and 49 respectively) of SARS-COV-2 (100 nM) and challenged 24h after with 600 TCID50 (¬0.1 MOI) of SARS-COV-2.

48 hours post-infection viral load was determined by endpoint dilution assay.

As seen from FIG. 15, the siRNA targeting NSP8 (SEQ ID NO: 7) and siRNA2 targeting RNA dependent RNA polymerase (SEQ ID NO: 48) caused a larger than 95% reduction in viral load. In particular, when a combination of the two siRNAs was utilized a more than 500-fold in viral load was observed.

### Example 7—Validation of siRNA Efficiency—Viral Load Assay

Vero E6 cells (known to be easily infected by SARS-COV-2) were transfected with siRNAs (S1, S3, S5, S9 SEQ ID NO:89, 91, 93 and 97) of Sars-COV2; the helicase (Hel_14—SEQ ID NO: 13) the Nsp8 protein (NSP8_8—SEQ ID NO: 7), the RNA dependent polymerase (Pol49- SEQ ID NO: 48), (100 nM) and after 24 h challenged with 6000 or 600 times the TCID50 of SARS-COV-2.

48 hours post-infection remaining virus was determined by endpoint dilution assay.

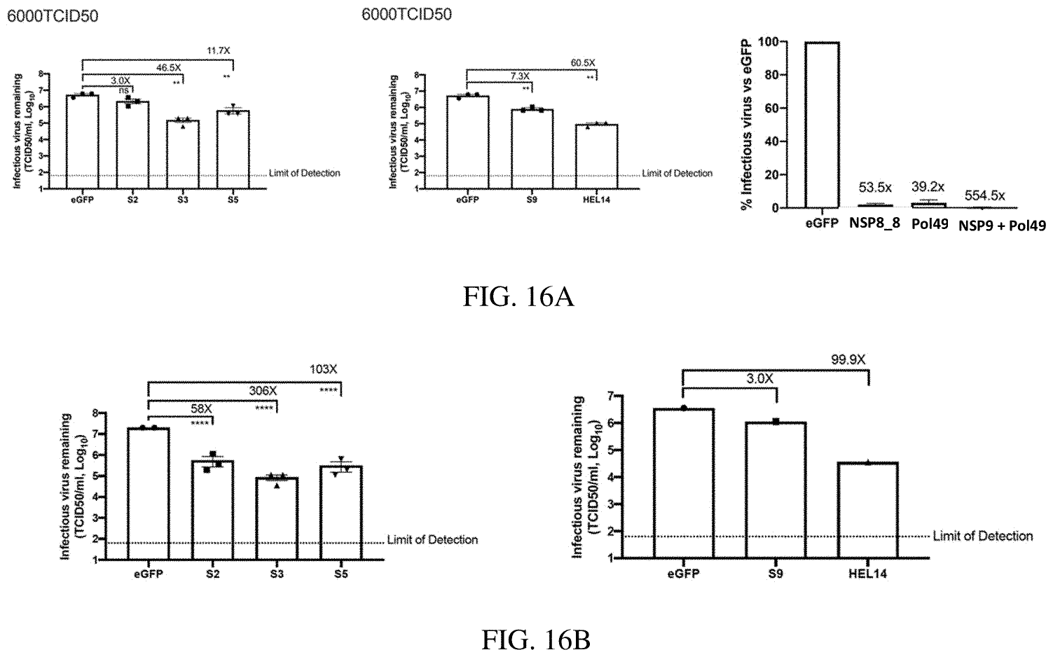

As seen from FIG. 16A and FIG. 16B, the all the tested siRNAs caused a significant reduction in remaining viruses, indicating that the siRNAs recovered in the screen can be used for attenuation (preventing and/or treating Sars-COV2 infection. Significantly, providing a cocktail of siRNAs including both NSP8_8 and Pol49) essentially eliminated the virus.

### Example 8—Validation of siRNA Cocktails

Vero E6 cells (known to be easily infected by SARS-COV-2) were transfected with a cocktail of siRNAs targeting various regions of the SARS-COV2 (100 nM) and after 48 h challenged with 6000, 600 or 60 times the TCID50 of SARS-COV-2.

48 hours post-infection remaining virus was determined by evaluation of depletion E-protein (blue/dark bars) and RNA dependent RNA polymerase (RDRP—orange/light bars) levels as compared to EGFP (indicative of transfection efficiency).

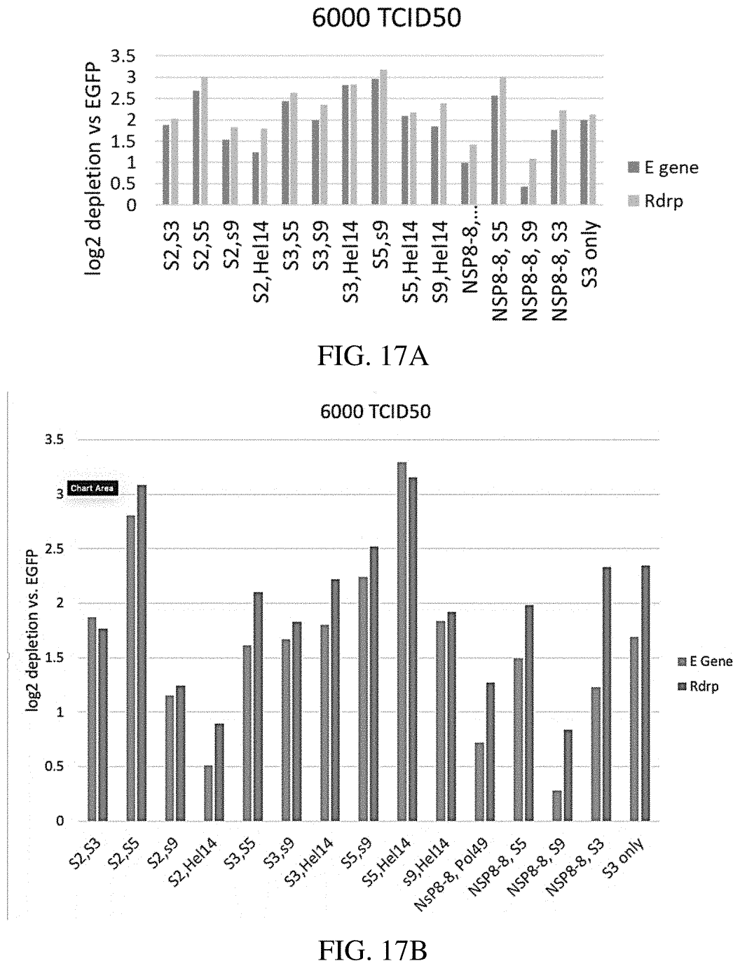

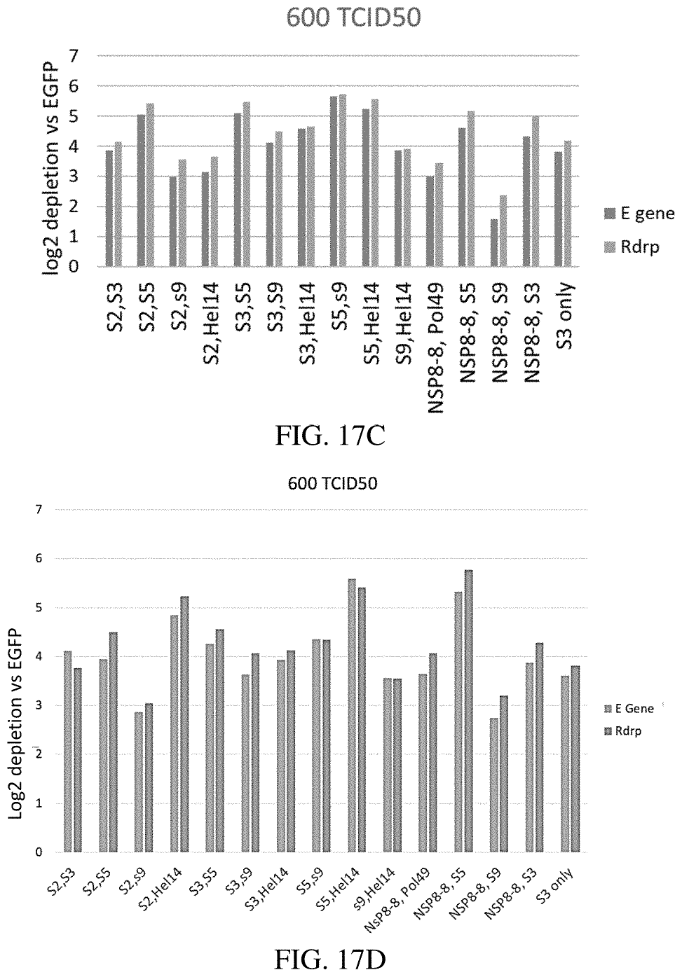

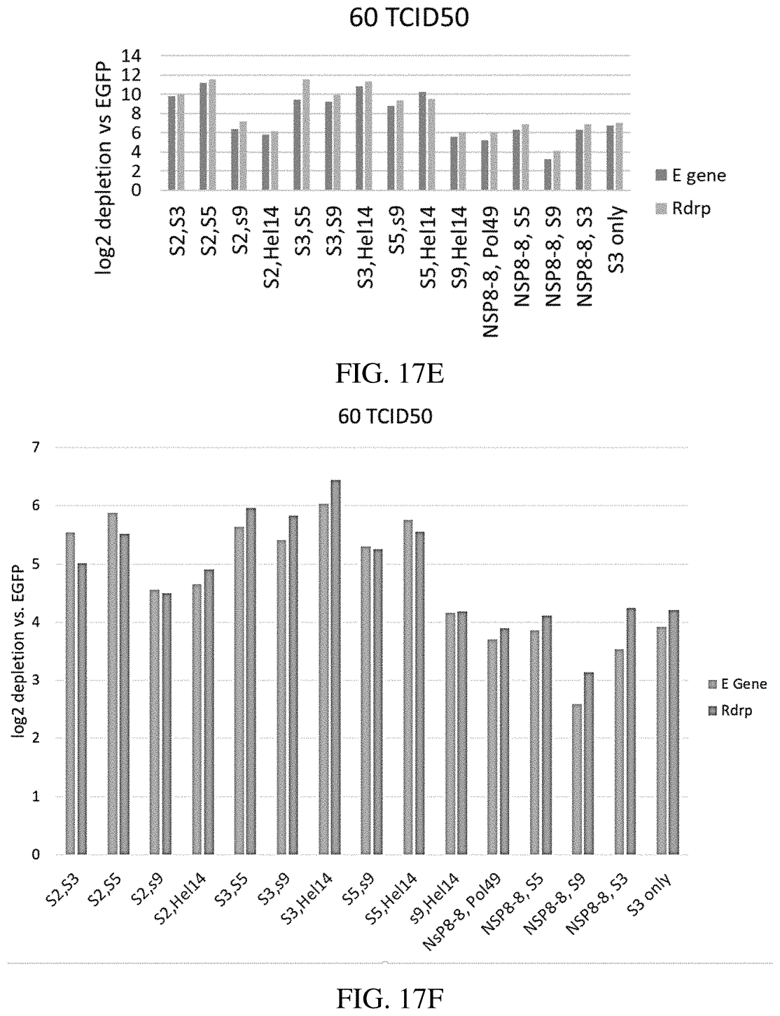

As seen from FIG. 17A-FIG. 17F, several cocktails, in particular cocktails containing siRNAs targeting S2 and S3 (SEQ ID Nos 90 and 91); S2 and S5 (SEQ ID NOs 90 and 93); S2 and S9 (SEQ ID NOs 90 and 97); S2 and Helicase (He114) (SEQ ID NOs 90 and 13); S5 and Helicase (Hel14) (SEQ ID NOs 93 and 13); and NSP8 and S9 (SEQ ID NOs: 7 and 97) were found to significantly increase virus depletion as compared to S3 only.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-43. (canceled)

44. An isolated ribonucleic acid interfering (RNAi) molecule, the RNAi molecule comprising a sequence of from about 15 to about 65 base pairs, capable of decreasing expression of a SARS-CoV-2 transcript, and wherein the RNAi molecule comprises at least one nucleotide sequence having at least 90% sequence homology to any one of SEQ ID NOs: 4, 6-9, 12, 13, 60, 83, 84, 90, 91, 93, 94 and 97.

45. The isolated RNAi molecule of claim 44, wherein the RNAi molecule comprises at least one nucleotide sequence having at least 90% sequence homology to any one of SEQ ID NO: 13; 90, 91 and 93.

46. The isolated RNAi molecule of claim 45, wherein the RNAi molecule comprises at least one nucleotide sequence having at least 90% sequence homology to any one of SEQ ID NO: 93.

47. The isolated RNAi molecule of claim 44, wherein a first strand and a second strand of the RNAi molecule comprise at least 14-25 contiguous complementary base pairs.

48. The isolated RNAi molecule of claim 47, wherein the first and the second strands comprise a 3′ single-stranded overhang.

49. The isolated RNAi molecule of claim 48, wherein the overhang comprises at least two nucleotides.

50. The isolated RNAi molecule of claim 47, wherein the first and/or the second strand comprises a nucleotide analogue.

51. The isolated RNAi molecule of claim 1, wherein the at least one nucleotide sequence has 100% sequence homology to any one of SEQ ID Nos: 4, 6-9, 12, 13, 60, 83, 84, 90, 91, 93, 94 and 97.

52. The isolated RNAi molecule of claim 1, wherein the RNAi molecule is a siRNA or a shRNA.

53. A composition comprising the one or more of the RNAi molecules of claim 44 and a suitable transport vehicle and/or carrier.

54. The composition of claim 53, comprising at least two different RNAi molecules, wherein the first strand of each of the at least two RNAi molecules has 90% sequence homology to different ones of any one of SEQ ID NO: 4, 6-9, 12, 13, 60, 83, 84, 90, 91, 93, 94 and 97.

55. The composition of claim 53, wherein at least one of the RNAi molecules has 90% sequence homology to SEQ ID NO: 90, 91, 93, 94 or 97.

56. The composition of claim 55, wherein at least one of the RNAi molecules is SEQ ID NO: 93.

57. The composition of claim 53, wherein the carrier is aqueous solution.

58. The composition of claim 53, wherein the composition is suitable for administration through aerosol.

59. The composition of claim 53, wherein the transport vehicle is a liposome or a lipid nanoparticle.

60. The composition of claim 53, formulated for oral and/or nasal administration, for administration via inhalation or for intranasal and/or intrabuccal administration.

61. A method for preventing infection, reducing viral load, attenuating and/or inhibiting spread of SARS-CoV-2 infection, the method comprising administering to a subject one or more of the RNAi molecules of claim 44.

62. The method of claim 61, wherein the subject is a subject infected with the SARS-CoV-2.

63. The method of claim 61, wherein the subject is a care-giver of a patient infected with the SARS-CoV-2 or suspected as being infected with SARS-CoV-2.
